(19) United States
(12) Patent Application Publication
(10) Pub. No.: US 2013/0317094 A1
(43) Pub. Date: Nov. 28, 2013

(54) USE OF ELLAGITANNINS AS INHIBITORS OF BACTERIAL QUORUM SENSING

(71) Applicant: THE FLORIDA INTERNATIONAL UNIVERSITY BOARD OF TRUSTEES, Miami, FL (US)

(72) Inventors: Kalai Mathee, Miami, FL (US); Allison L. Adonizio, Cambridge, MA (US); Frederick Ausubel, Boston, MA (US); Jon Clardy, Boston, MA (US); Bradley Bennett, Miami, FL (US); Kelsey Downum, Arlington, TX (US)

(73) Assignee: THE FLORIDA INTERNATIONAL UNIVERSITY BOARD OF TRUSTEES, Miami, FL (US)

(21) Appl. No.: 13/893,916

(22) Filed: May 14, 2013

Related U.S. Application Data
(63) Continuation of application No. 12/922,555, filed on Dec. 14, 2010, now abandoned.
(60) Provisional application No. 61/036,812, filed on Mar. 14, 2008.

(51) Int. Cl. A01N 43/24; A61K 31/357; A61K 45/06

(52) U.S. Cl. CPC A01N 43/24; A61K 45/06; A61K 31/357
USPC 514/450

(57) ABSTRACT

Described herein are materials and methods for the inhibition of bacterial QS. Methods of treating bacterial infections by administration of one or more ellagitannins in amount effective to inhibit bacterial QS is also provided.

### CROSS-REFERENCE TO RELATED APPLICATION

The present application claims the benefit of priority of U.S. Provisional Application No. 61/036,812, filed Mar. 14, 2008, the disclosure of which is incorporated herein by reference in its entirety.

### FIELD OF THE INVENTION

The present application is directed to the use of ellagitannins for the inhibition of bacterial quorum sensing.

### BACKGROUND OF THE INVENTION

Many microbial pathogens cause tremendous damage worldwide, in humans as well as in animals and crop plants. The continuing emergence of multiple-drug-resistant pathogen strains has necessitated finding new compounds that can be used in antimicrobial treatment. In general, two strategies exist for controlling pathogens, either kill the pathogen or attenuate its virulence such that it does not damage the host.

Many bacteria use autoinducer ligands to monitor their population densities in a phenomenon called quorum sensing. See Fuqua & Greenberg, Nature Reviews Molecular Cell Biology, 3:685-695, 2002; or de Kievit et al., Infection & Immunity, 68: 4839-4849, 2000, for a review of the Quorum sensing system in pathogenic bacteria. Bacteria use quorum sensing to regulate a variety of phenotypes, such as biofilm formation, toxin production, exopolysaccharide production, virulence factor production, and motility, which are essential for the successful establishment of a symbiotic or pathogenic relationship with their respective eukaryotic hosts (Marketon et al., J. Bacteriol., 185:325-331, 2003; Ohtani et al., Mol. Microbiol., 44:171-179, 2002; Quinones et al., Mol. Plant-Microbe Interact, 18:682-693, 2005; Rice et al., J. Bacteriol., 187:3477-3485, 2005; Suntharalingam et al., Trends Microbiol., 13:3-6, 2005). At high cell densities, bacteria use this chemical signaling process to switch from a nomadic existence to that of a multicellular community. This lifestyle switch is significant, as numerous pathogenic bacteria use quorum sensing to turn on virulence pathways and form drug-impervious communities called biofilms that are the basis of a myriad chronic infections. Over 80% of bacterial infections in humans involve the formation of biofilms, as exemplified in lung infections by Pseudomonas aeruginosa, which is the primary cause of morbidity in cystic fibrosis patients. The treatment of infections by pathogens that form biofilms costs over $1 billion/year in the US alone. Studies with animal models have shown that strains with inactivated quorum sensing genes show reduced virulence. Disrupting quorum sensing may interfere with the ability of bacteria to form robust biofilms and thus render the bacteria more sensitive to antibacterial agents and the host's immune response.

Quorum sensing is mediated by a signal molecule that binds to a cognate transcriptional activator to cause either upregulation or repression of genes that increase virulence factors, which include exotoxins, proteases, alginates, lipopolysaccharides, pyocyanin and rhamnolipids. At low bacterial cell density, the concentration of the signaling molecule does not activate the virulence genes, while at higher bacterial density, the concentration of the signaling molecule reaches a critical threshold to activate virulence genes.

In Gram negative bacteria, for example, the signal molecule is an acylated homoserine lactone (AHSL), often referred to as the autoinducer, which interacts with a protein of a quorum regulon. A quorum regulon includes two proteins, the autoinducer synthase (the I protein) and the regulator (the R protein), which, upon binding of the autoinducer, activates the transcription of numerous genes. In Pseudomonas aeruginosa, two quorum regulons have been identified. One quorum regulon is known as the LasIR system and is mediated by a 3-oxo-dodecanoyl homoserine lactone (3-oxo-C12-HSL) signal molecule. The other quorum regulon is known as the RhlIR system and is mediated by a butyryl homoserine lactone (C4-HSL) signal molecule.

In recent years it has become apparent that many Gram-negative bacteria employ one or more quorum sensing systems. The quorum sensing system is an attractive anti-bacterial target because it is not found in humans and is critical for high level bacterial virulence. Bacterial quorum sensing systems comprise AHL derivatives with different acyl side chains to regulate, in a cell-density dependent manner, a wide variety of physiological processes unique to the life-cycle of each microbe. These processes include: swarming, motility, biofilm formation, conjugation, bioluminescence and/or production of pigments, antibiotics and enzymes. For example, in P. aerugniosa quorum sensing pathways affect the expression of various exoenzymes, biofilm formation and cell-cell spacing. Other bacteria react to quorum sensing stimulation by expressing proteases and pectinases, expressing pili, entering stationary phase, emerging from lag phase and initiating cell division.

Biofilms are dense extracellular polymeric matrices in which the bacteria embed themselves. Biofilms allow bacteria to create a microenvironment that attaches the bacteria to the host surface and which contains excreted enzymes and other factors allowing the bacteria to evade host immune responses including antibodies and cellular immune responses. Such biofilms can also exclude antibiotics. Further, biofilms can be extremely resistant to removal and disinfection. For individuals suffering from cystic fibrosis, the formation of biofilms by P. aerugniosa is eventually fatal. Other bacteria also respond to quorum sensing signals by producing biofilms. Biofilms are inherent in dental plaques, and are found on surgical instruments, food processing and agriculture equipment and water treatment and power generating machinery and equipment.

Because of the virulence factors it triggers, the bacterial quorum-sensing system offers a target for use in modulating the virulence of pathogenic bacteria. All acyl-homoserine lactone quorum-sensing systems described to date, except that of V. harveyi, utilize AI synthases encoded by a gene homologous to luxI of V. fischeri. The response to the autoinducer is mediated by a transcriptional activator protein encoded by a gene homologous to luxR of V. fischeri (Bassler and Silverman, in Two Component Signal Transduction, Hoch et al., eds., Am. Soc. Microbiol. Washington D.C., pp. 431-435, 1995).

Gram-negative bacteria represent numerous relevant pathogens using quorum-sensing pathways. Besides P. aeruginosa, other gram-negative quorum sensing bacteria include: Aeromonas hydrophila, A. salmonicida, Agrobacterium tumefaciens, Burkholderia cepacia, Chromobacterium violaceum, Enterobacter agglomeran, Erwinia carotovora, E. chrysanthemi, Escherichia coli, Nitrosomas europaea, Obesumbacterium proteus, Pantoea stewartii, Pseudomonas aureofaciens, P. syringae, Ralstonia solanacearum, Rhisobium etli, R. leguminosarum, Rhodobacter sphaeroides, Serratia liguefaciens, S. marcescens, Vibrio anguillarum, V. fischeri, V. cholerae, Xenorhabdus nematophilus, Yersinia enterocolitica, Y. pestis, Y. pseudotuberculosis, Y. medievalis, and Y. ruckeri.

In addition to their pathogenic costs, quorum sensing bacteria also have significant economic impact in industries other than health care. For example, in agriculture, various species of the genera Rhizobium, Bradyrhizobium and Sinorhizobium are important plant symbionts helping legumes to fix nitrogen, while, species of the genera Erwinia, Xanthomonas and Pseudomonas are responsible for significant food-spoilage. Other industries, such as power generation, paper making and water treatment are subject to biofouling by many types of slime forming bacteria, such as Deinococcus geothermalis.

Tannins are widespread throughout the angiosperms (Okuda et al., Phytochem., 32:507-521, 1993), conferring structural benefits to the plant while providing protection through antioxidant and anti-feedant activity. Often classified as “waste” in natural products chemistry due to their abundance and lack of protein specificity (Zhu et al., 1997), tannins and other polyphenolics have been previously ignored by the pharmaceutical industry.

Many polyphenolics possess antimicrobial activity potentially explained by inhibition of microbial enzymes, substrate or iron deprivation, or inhibition of oxidative phosphorylation (Scalbert, Phytochem., 30:3875-3883, 1991). However, the same study shows most bacteria are not susceptible to ellagitannins, i.e. these compounds do not seem to have growth inhibition or cidal effects. Furthermore, it has been shown that ellagic acid (a component of ellagitannins) can interfere with bacterial quorum sensing (Huber et al., Biosciences, 58:879-884, 2004).

Thus, there is a need to identify and develop compounds that are useful as inhibitors of bacterial quorum sensing.

### SUMMARY OF THE INVENTION

The present application is based on the discovery that ellagitannins, components in some medicinal plants, are capable of inhibiting quorum sensing (QS) in pathogenic bacteria. Thus, in one aspect, the invention provides methods of inhibiting QS in pathogenic bacteria in a mammalian subject contacting the bacteria with an ellagatannin in an amount effective to inhibit QS in the bacteria. In one embodiment, the bacteria is contacted with the ellagitannin in vivo. In such embodiments, the contacting comprises administering the ellagitannin to the mammalian subject. In one embodiment, the mammalian subject is afflicted with a bacterial infection associated with bacterial QS and the ellagitannin is administered in an amount effective to treat the bacterial infection. In another embodiment, the mammalian subject is afflicted with a disorder associated with biofilm formation and the ellagitannin is administered in an amount effective to treat the disorder. In one embodiment, the mammalian subject is human. In another embodiment, the human is immunocompromised (e.g., having, for example and without limitation, cancer or AIDS).

Another aspect of the invention provides a method of treating a bacterial infection associated with QS in a mammalian subject, the method comprising administering to the subject an ellagitannin in an amount effective to treat the infection. In one embodiment, the infection an infection caused by a bacterium is selected from the group consisting of: Aeromonas hydrophila, Aeromonas salmonicida, Agrobacterium tumefaciens, Burkholderia cepacia, Chromobacterium violaceum, Enterobacter agglomeran, Erwinia carotovora, Erwinia chrysanthemi, Escherichia coli, Nitrosomas europaea, Obesumbacterium proteus, Pantoea stewartii, Pseudomonas aureofaciens, Pseudomonas aeruginosa, Pseudomonas syringae, Ralstonia solanacearum, Rhisobium etli, Rhisobium leguminosarum, Rhodobacter sphaeroides, Serratia liguefaciens, Serratia marcescens, Staphylococcus aureus, Staphyllococcus epidermidis, Vibrio anguillarum, Vibrio fischeri, Vibrio cholerae, Xenorhabdus nematophilus, Yersinia enterocolitica, Yersinia pestis, Yersinia pseudotuberculosis, Yersinia medievalis, and Yersinia ruckeri. In some embodiments, exemplary bacterial infections include, but are not limited to, bacteremia, septicemia, endo- and pericarditis, sinusitis, upper respiratory tract infection, chronic bronchitis, pneumonia, cerebral and pulmonary lesions, meningitis, dermatitis or folliculitis, necrotizing fascitis, cellulitis, urinary tract infections, osteomylitis, enterocolitis, contact lens-associated kerititis and conjunctivitis. In some embodiments, the mammalian subject to be treated is an immunocompromised individual, such as a human subject, for example and without limitation, having cancer or AIDS.

In another aspect, the present invention provides a method of treating a disorder associated with biofilm formation in a mammalian subject, the method comprising administering an ellagitannin to the subject in an amount effective to disrupt biofilm formation in the subject. In various embodiments, the disorder associated with biofilm formation in the subject is selected from the group consisting of cystic fibrosis, dental caries, periodonitis, otitis media, muscular skeletal infections, necrotizing fasciitis, biliary tract infection, osteomyelitis, bacterial prostatitis, endocarditis, native valve endocarditis, cystic fibrosis pneumonia, meloidosis, or skin lesions associated with bullous impetigo, atopic dermatitis and pemphigus foliaceus or implanted device-related infections. In some embodiments, the condition is a nosocomial infection, including but not limited to, pneumonia or an infection associated with sutures, exit sites, arteriovenous sites, scleral buckles, contact lenses, urinary catheter cystitis, peritoneal dialysis (CAPD) peritonitis, IUDs, endotracheal tubes, Hickman catheters, central venous catheters, mechanical heart valves, vascular grafts, biliary stent blockage, and orthopedic devices.

Also provided is a method of modulating biofilm formation on a surface, the method comprising contacting the surface with an ellagitannin in an amount effective for disrupt or inhibit biofilm formation on the surface. In one embodiment, the surface is an inanimate surface. Exemplary inanimate surfaces include, but are not limited to, metal, glass, plastic, wood and stone surfaces. In another embodiment, the surface is an animate surface. Exemplary animate surfaces include, but are not limited to, mammalian tissues, mammalian membranes, mammalian skin.

As used herein, the term “pathogenic bacterium” or “pathogenic bacteria” refers to both gram-negative and gram-positive bacterial cells capable of infecting and causing disease in a mammalian host, as well as producing infection-related symptoms in the infected host, such as fever or other signs of inflammation, intestinal symptoms, respiratory symptoms, dehydration, and the like.

In some embodiments, and without limitation, the bacteria is of a genus selected from the group consisting of Aeromonas, Agrobacterium, Burkholderia, Chromobacterium, Enterobacter, Erwinia, Escherichia, Nitrosomas, Obesumbacterium, Pantoea, Pseudomonas, Ralstonia, Rhisobium, Rhodobacter, Serratia, Staphyllococcus, Vibrio, Xenorhabdus, and Yersinia. For example, in some embodiments and without limitation, the bacteria is of a species selected from the group consisting of Aeromonas hydrophila, Aeromonas salmonicida, Agrobacterium tumefaciens, Burkholderia cepacia, Chromobacterium violaceum, Enterobacter agglomeran, Erwinia carotovora, Erwinia chrysanthemi, Escherichia coli, Nitrosomas europaea, Obesumbacterium proteus, Pantoea stewartii, Pseudomonas aureofaciens, Pseudomonas aeruginosa, Pseudomonas syringae, Ralstonia solanacearum, Rhisobium etli, Rhisobium leguminosarum, Rhodobacter sphaeroides, Serratia liguefaciens, Serratia marcescens, Staphylococcus aureus, Staphyllococcus epidermidis, Vibrio anguillarum, Vibrio fischeri, Vibrio cholerae, Xenorhabdus nematophilus, Yersinia enterocolitica, Yersinia pestis, Yersinia pseudotuberculosis, Yersinia medievalis, and Yersinia ruckeri.

Also provided is a method of treating a disorder associated with QS in a mammalian subject resistant to treatment with a standard of care anti-bacterial therapeutic comprising administering to the subject an ellagitannin in an amount effective to inhibit QS in the bacteria causing the infection.

In some embodiments, the methods described herein further comprise the step of administering a standard of care anti-bacterial therapeutic to the subject in need of treatment. In the context of methods of the invention, “standard of care” refers to a treatment that is generally accepted by clinicians for a certain type of patient diagnosed with a type of illness. For cardiac disorders, for example, an aspect of the invention is to improve standard of care therapy with co-therapy with one or more ellagitannins described herein. Exemplary standard of care anti-bacterial therapeutics include, but are not limited to, colloidal silver, penicillin, penicillin G, erythromycin, polymyxin B, viomycin, chloromycetin, streptomycins, cefazolin, ampicillin, methicillin, oxacillin, nafcillin, cloxacillin, dicloxacillin azactam, tobramycin, cephalosporins (including cephalothin, cefazolin, cephalexin, cephradine, cefamandole, cefoxitin, and 3rd-generation cephalosporins), Carbapenems (including imipenem, meropenem, Biapenem), bacitracin, tetracycline, doxycycline, gentamycin, quinolines, neomycin, clindamycin, kanamycin, metronidazole, treptogramins (including Quinupristin/dalfopristun (Synercid™)), Streptomycin, Ceftriaxone, Cefotaxime, Rifampin, Glycopeptides (including vancomycin, teicoplanin, LY-333328 (Ortivancin)), Macrolides (including erythromycin, clarithromycin, azithromycin, lincomycin, and clindamycun), Ketolides (including Telithromycin, ABT-773), Tetracyclines, Glycylcyclines (including Terbutyl-minocycline (GAR-936)), Aminoglycosides, Chloramphenicol, Imipenem-cilastatin, Glycopeptides (including oritavancin, LY-333328, dalbavancin), Fluoroquinolones (including ofloxacin, sparfioxacin, gemifloxacin, cinafloxacun (DU-6859a)) and other topoisomerase inhibitors, Trimethoprim-sulfamethoxazole (TMP-SMX), Ciprofloxacin, topical mupirocin, Oxazolidinones (including AZD-2563, Linezolid (Zyvox™)), Lipopeptides (including Daptomycin, Ramoplanin), ARBELIC (TD-6424) (Theravance), TD-6424 (Theravance), isoniazid (INN), rifampin (RIF), pyrazinamide (PZA), Ethambutol (EMB), Capreomycin, cycloserine, ethionamide (ETH), kanamycun, and p-aminosalicylic acid (PAS).

Combination therapy comprising an ellagitannin and a standard of care anti-bacterial therapeutic described herein for the treatment of a bacterial infection associated with QS is specifically contemplated. For example, in one embodiment, the invention provides a method of treating a bacterial infection associated with bacterial QS in a mammalian subject in need of treatment comprising administering to the subject a therapeutically-effective amount of a combination therapy comprising (a) an ellagitannin and (b) a standard of care anti-bacterial therapeutic. In another embodiment, the invention provides a method of treating a disorder associated with biofilm formation in a mammalian subject comprising administering to the subject a therapeutically-effective amount of a combination therapy comprising (a) an ellagitannin and (b) a standard of care anti-bacterial therapeutic. In another embodiment, the invention provides a method of treating a disorder associated with bacterial QS (or biofilm formation) in a mammalian subject comprising administering to the subject a therapeutically-effective amount of a combination therapy comprising (a) an ellagitannin and (b) a standard of care additional/second agent as described herein.

Such combination therapy would be provided in a combined amount effective to inhibit QS in the bacteria and/or treat the bacterial infection and/or treat the disorder associated with biofilm formation. This process involves administering to a subject in need thereof an ellagitannin and a standard of care anti-bacterial therapeutic at the same time, which may be achieved by administering a single composition or pharmacological formulation that includes both an ellagitannin and a standard of care therapeutic, or by administering two distinct compositions or formulations, at the same time, wherein one composition includes an ellagitannin and the other includes a standard of care anti-bacterial therapeutic. In another embodiment, the combination therapy involves administering to a subject in need thereof an ellagitannin and a standard of care anti-bacterial therapeutic at different times, which may be achieved by administering two distinct compositions or formulations, at different time intervals, wherein one composition includes an ellagitannin and the other includes a standard of care anti-bacterial therapeutic.

In some embodiments, the ellagitannin is selected from the group consisting of vescalagin, castalagin, punicalin, rhoipteleanin H, rhoipteleanin I, rhoipteleanin J, tellimagrandin I, tellimagrandin II (eugeniin), pterocaryanin C, sanguin H-4, sanguin H-5, casuarictin, potentillin, hemicetal congener pedunculagin, davidiin, corilagin, geraniin, carpinusin, chebulinic acid, chebulagic acid, elaeocarpusin, repandusinic acid A, repandusinin, stachyurin, casuarinin, pedunculagin, 5-desgalloyl-stachyurin, casuariin, roburin A, roburin D, cercidinin A, cercidinin B, cuspinin, platycaryanin D, nupharin A, sanguiin H-6, grandinin, coriariin, agrimoniin, rugosin D, oenothein B, woodfordin C, strictinin and trapanin B. In one embodiment, the ellagitannin is vescalagin. In another embodiment, the ellagitannin is castalagin.

Compositions comprising the ellagitannin and a pharmaceutically-acceptable carrier, diluent or excipient are also contemplated.

Also provided is the use of an ellagitannin in the manufacture of a medicament for the treatment of a disorder associated with bacterial QS (or for treatment of a disorder associated with biofilm formation).

The foregoing summary is not intended to define every aspect of the invention, and additional aspects are described in other sections, such as the Detailed Description. The entire document is intended to be related as a unified disclosure, and it should be understood that all combinations of features described herein may be contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs herein. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus.

It should be understood that while various embodiments in the specification are presented using “comprising” language, under various circumstances, a related embodiment may also be described using “consisting of” or “consisting essentially of” language. It is to be noted that the term “a” or “an”, refers to one or more, for example, “an ellagitannin,” is understood to represent one or more ellagitannins. As such, the terms “a” (or “an”), “one or more,” and “at least one” can be used interchangeably herein.

### BRIEF DESCRIPTION OF THE FIGURES

FIG. 1 shows thin layer chromatography (TLC) of C. erectus crude extract and the visualization of phenolic anti-QS activity.

FIG. 2 is a schematic of fractionation of C. erectus crude extract.

FIG. 3 shows the results of an anti-QS bioassay of fractionation products of C. erectus.

FIG. 4 shows the HPLC separation of Fraction A.

### DETAILED DESCRIPTION OF THE INVENTION

Many bacterial phenotypic traits are modulated in response to bacterial density that is detected by QS. These phenotypes have important health consequences in pathogenic bacteria and include virulence, carbapenem antibiotic production, biofilm formation, enzyme synthesis and secondary metabolite synthesis. Modulation or interruption of these signaling pathways can alter the life-cycle of quorum-sensing bacteria and thereby alter their virulence.

A number of medicinal plants, including Conocarpus erectus, have been found to be effective in inhibiting the pathogenicity of P. aeruginosa via attenuation of the QS system (Adonizio et al., 2008a; Adonizio et al., 2008b; Adonizio et al., 2006, the disclosures of which are incorporated herein by reference in their entireties), but prior to the filing of the present application, the active components responsible for the inhibition of QS was not known.

Commonly known as buttonwood, C. erectus has been used throughout the Caribbean, Puerto Rico, and parts of Africa against catarrh, conjunctivitis, diarrhea, syphilis, and gonorrhea (Melendez, 1982, the disclosure of which is incorporated herein by reference in their entireties). The activity of this plant on the bacterial QS system may explain its traditional use for these diseases. The data presented herein identified two hydrolyzable tannins, vescalagin and castalagin, to be responsible for anti-QS activity in C. erectus. Thus, the use of ellagitannins as an inhibitor of QS activity is specifically contemplated.

I. Ellagitannins

The term “ellagitannin” as used herein means a compound having a polyol core that is esterified with at least two galloyl moieties, wherein at least two of the galloyl moieties are oxidatively carbon-carbon coupled to each other. In one embodiment, the polyol core is a carbohydrate. In another embodiment, the polyol core is glucose. In another embodiment, the polyol core is D-glucose. In yet another embodiment, the polyol core is an open-chain D-glucose. When the ellagitannin comprises a carbohydrate polyol core, the anomeric carbon can form a C- or O-glycosidic bond with a galloyl moiety. In one embodiment, the ellagitannin forms a C-glycosidic bond with the galloyl moiety.

In some embodiments, the carbon-carbon coupled galloyl moieties are 4,6-hexahydroxybiphenoyl (HHBP or castalagin) and/or 2,3,5-nonahydroxyterphenoyl (NHTP or vescalagin).

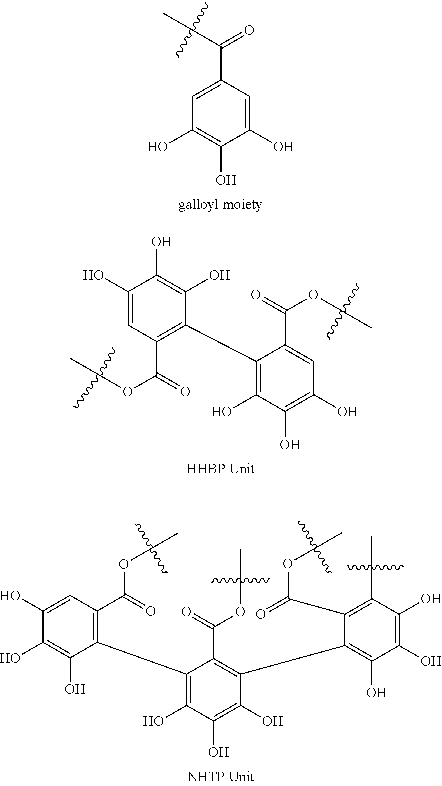 [SMILES: *C(=O)c1cc(O)c(O)c(O)c1 ; *OC(=O)c1cc(O)c(O)c(O)c1-c1c(C(=O)O*)cc(O)c(O)c1O ; *OC(=O)c1cc(O)c(O)c(O)c1-c1c(O)c(O)c(O)c(-c2c(O)c(O)c(O)c(*)c2C(=O)O*)c1C(=O)O*]

In one embodiment, the ellagitannin comprises a C-glycosidic, open-chain D-glucose core coupled to HHBP and NHTP (e.g. castalagin and vescalagin, respectively).

Castalagin and vescalagin (Mayer et al., 1967; Mayer et al., 1970) belong to a sub-class of hydrolyzable tannins known as C-glycosidic ellagitannins derived from gallic acid metabolism (Quideau & Feldman, 1996). Castalagin and vescalagin are highly water-soluble compounds featuring an open-chain glucose core esterified to numerous oxidatively coupled galloyl moieties (specifically a 4,6-hexahydroxybiphenoyl (HHBP) unit and a 2,3,5-nonahydroxyterphenoyl (NHTP) unit) (Khanbabaee & van Ree, 2001). These complex structural units confer stereochemical rigidity to the molecule, and in fact, C-glycosidic ellagitannins would seem to have a higher tendency for selective protein interaction than other classes of polyphenolics (Haslam, 1996; Zhu et al., 1997).

Ellagitannins known in the art, other than castalagin and vescalagin, are also contemplated for use in the methods described herein. Such ellagitannins include, but are not limited to, punicalin [4,6-(S—S)-gallagyl-D-glucopyranose], Rhoipteleanin H, Rhoipteleanin I, Rhoipteleanin J, tellimagrandin I, tellimagrandin II (eugeniin), pterocaryanin C, sanguine H-4, sanguine H-5, casuarictin, potentillin, hemicetal congener pedunculagin, davidiin, corilagin, geraniin, carpinusin, chebulinic acid, chebulagic acid, elaeocarpusin, repandusinic acid A, repandusinin, stachyurin, casuarinin, pedunculagin, 5-desgalloyl-stachyurin, casuariin, roburin A, roburin D, cercidinin A, cercidinin B, cuspinin, platycaryanin D, nupharin A, sanguiin H-6, grandinin, coriariin, agrimoniin, rugosin D, oenothein B, woodfordin C, strictinin and trapanin B. For a review of ellagitannins, see Quideau et al., Chem. Rev., 96:475-504, 1996 and Khanbabaee et al., Nat. Prod. Rep., 18:641-649, 2001).

II. Therapeutic Uses of Ellagitannins

The invention provides in one aspect a method of inhibiting bacterial QS comprising contacting the bacteria with an ellagitannin in an amount effective to inhibit QS in the bacteria. In one embodiment, the bacteria is contacted with the ellagitannin in vivo. In such embodiments, the contacting comprises administering the ellagitannin to the mammalian subject. In one embodiment, the mammalian subject is afflicted with a bacterial infection associated with bacterial QS and the ellagitannin is administered in an amount effective to treat the bacterial infection. In another embodiment, the mammalian subject is afflicted with a disorder associated with biofilm formation and the ellagitannin is administered in an amount effective to treat the disorder. In one embodiment, the mammalian subject is human. In another embodiment, the human is immunocompromised (e.g., having, for example and without limitation, cancer or AIDS). Practice of methods of the invention in other mammalian subjects, especially mammals that are conventionally used as models for demonstrating therapeutic efficacy in humans (e.g., primate, porcine, canine, or rabbit animals), is also contemplated.

In another embodiment, the bacteria is contacted with the ellagitannin ex vivo. In such an embodiment, for example, the contacting comprises administering the ellagitannin to a surface in an amount effective to inhibit biofilm formation associated with bacterial quorum sensing on surface (including without limitation, a medical device).

In another aspect, the invention provides a method of treating a bacterial infection associated with QS in a mammalian subject comprising administering to the subject one or more ellagitannins in an amount effective to inhibit QS in the bacteria.

In yet another aspect, the invention provides a method of treating a disorder associated with biofilm formation in a mammalian subject are also provided. Such methods comprise administering one or more ellagitannins to the subject in an amount effective to disrupt biofilm formation in the subject.

In one embodiment, the disorder associated with biofilm formation in the subject is selected from the group consisting of cystic fibrosis, dental caries, periodonitis, otitis media, muscular skeletal infections, necrotizing fasciitis, biliary tract infection, osteomyelitis, bacterial prostatitis, endocarditis, native valve endocarditis, cystic fibrosis pneumonia, meloidosis, or skin lesions associated with bullous impetigo, atopic dermatitis and pemphigus foliaceus or implanted device-related inventions. In another embodiment, the condition is a nosocomial infection, including but not limited to, pneumonia or an infection associated with sutures, exit sites, arteriovenous sites, scleral buckles, contact lenses, urinary catheter cystitis, peritoneal dialysis (CAPD) peritonitis, IUDs, endotracheal tubes, Hickman catheters, central venous catheters, mechanical heart valves, vascular grafts, biliary stent blockage, and orthopedic devices.

In some embodiments and without limitation, the bacteria is of a genus selected from the group consisting of Aeromonas, Agrobacterium, Burkholderia, Chromobacterium, Enterobacter, Erwinia, Escherichia, Nitrosomas, Obesumbacterium, Pantoea, Pseudomonas, Ralstonia, Rhisobium, Rhodobacter, Serratia, Staphylococcus, Vibrio, Xenorhabdus, and Yersinia. For example, in some embodiments and without limitation, the bacteria is of a species selected from the group consisting of Aeromonas hydrophila, Aeromonas salmonicida, Agrobacterium tumefaciens, Burkholderia cepacia, Chromobacterium violaceum, Enterobacter agglomeran, Erwinia carotovora, Erwinia chrysanthemi, Escherichia coli, Nitrosomas europaea, Obesumbacterium proteus, Pantoea stewartii, Pseudomonas aureofaciens, Pseudomonas aeruginosa, Pseudomonas syringae, Ralstonia solanacearum, Rhisobium etli, Rhisobium leguminosarum, Rhodobacter sphaeroides, Serratia liguefaciens, Serratia marcescens, Staphyllococcus aureus, Vibrio anguillarum, Vibrio fischeri, Vibrio cholerae, Xenorhabdus nematophilus, Yersinia enterocolitica, Yersinia pestis, Yersinia pseudotuberculosis, Yersinia medievalis, and Yersinia ruckeri.

In various embodiments, an ellagitannin for use in the methods described is selected from the group consisting of vescalagin, castalagin, punicalin, Rhoipteleanin H, Rhoipteleanin I, Rhoipteleanin J, tellimagrandin I, tellimagrandin II (eugeniin), pterocaryanin C, sanguine H-4, sanguine H-5, casuarictin, potentillin, hemicetal congener pedunculagin, davidiin, corilagin, geraniin, carpinusin, chebulinic acid, chebulagic acid, elaeocarpusin, repandusinic acid A, repandusinin, stachyurin, casuarinin, pedunculagin, 5-desgalloyl-stachyurin, casuariin, roburin A, roburin D, cercidinin A, cercidinin B, cuspinin, platycaryanin D, nupharin A, sanguiin H-6, grandinin, coriariin, agrimoniin, rugosin D, oenothein B, woodfordin C, trapanin B and any combination thereof. In one embodiment, the ellagitannin is castalagin. In another embodiment, the ellagitannin is vescalagin.

In one embodiment, the methods described herein further comprise the step of administering a standard of care anti-bacterial therapeutic to the subject in need of treatment. In the context of methods of the invention, “standard of care” refers to a treatment that is generally accepted by clinicians for a certain type of patient diagnosed with a type of illness. For bacterial infections associated with bacterial QS, for example, an aspect of the invention is to improve standard of care therapy with co-therapy with one or more ellagitannins. Exemplary standard of care anti-bacterial therapeutics include, but are not limited to, colloidal silver, penicillin, penicillin G, erythromycin, polymyxin B, viomycin, chloromycetin, streptomycins, cefazolin, ampicillin, methicillin, oxacillin, nafcillin, cloxacillin, dicloxacillin azactam, tobramycin, cephalosporins (including cephalothin, cefazolin, cephalexin, cephradine, cefamandole, cefoxitin, and 3rd-generation cephalosporins), carbapenems (including imipenem, meropenem, Biapenem), bacitracin, tetracycline, doxycycline, gentamycin, quinolines, neomycin, clindamycin, kanamycin, metronidazole, treptogramins (including Quinupristin/dalfopristun (Synercid™)), Streptomycin, Ceftriaxone, Cefotaxime, Rifampin, glycopeptides (including vancomycin, teicoplanin, LY-333328 (Ortivancin), dalbavancin), macrolides (including erythromycin, clarithromycin, azithromycin, lincomycin, and clindamycun), ketolides (including Telithromycin, ABT-773), tetracyclines, glycylcyclines (including Terbutyl-minocycline (GAR-936)), aminoglycosides, chloramphenicol, Imipenem-cilastatin, fluoroquinolones (including ofloxacin, sparfioxacin, gemifloxacin, cinafloxacun (DU-6859a)) and other topoisomerase inhibitors, Trimethoprim-sulfamethoxazole (TMP-SMX), Ciprofloxacin, topical mupirocin, Oxazolidinones (including AZD-2563, Linezolid (Zyvox™)), Lipopeptides (including Daptomycin, Ramoplanin), ARBELIC (TD-6424) (Theravance), TD-6424 (Theravance), isoniazid (INN), rifampin (RIF), pyrazinamide (PZA), Ethambutol (EMB), Capreomycin, cycloserine, ethionamide (ETH), kanamycun, and p-aminosalicylic acid (PAS).

Also provided is a method of modulating biofilm formation on a surface, the method comprising contacting the surface with an ellagitannin in an amount effective for affecting biofilm formation on the surface. In one embodiment, the surface is an inanimate surface. Exemplary inanimate surfaces include, but are not limited to, metal, glass, plastic, wood and stone surfaces. In another embodiment, the surface is an animate surface. Exemplary animate surfaces include mammalian tissues, mammalian membranes, mammalian skin.

A. Combination Therapy

Combination therapy comprising one or more ellagitannins and a standard of care anti-bacterial therapeutic described herein for the treatment of a bacterial infection associated with QS is specifically contemplated. For example, in one aspect, the invention provides a method of treating a bacterial infection associated with bacterial QS in a mammalian subject in need of treatment comprising administering to the subject a therapeutically-effective amount of a combination therapy comprising (a) one or more ellagitannins and (b) a standard of care anti-bacterial therapeutic.

In other embodiments, the combination of an ellagitannin with one or more additional therapeutics/second agents in methods of the invention may reduce the amount of either agent needed as a therapeutically effective dosage, and thereby reduce any negative side effects the agents may induce in vivo. Exemplary additional therapeutic/second agents include, but are not limited to, dornase alfa (Pulmozyme®), CTFR-correcting drugs (including but not limited to, gentamicin), anti-inflammatory agents, NSAIDS, aldosterone antagonists, anti-bacterial agents, a COX-2 inhibitors, an α-adrenergic antagonist, an β-adrenergic antagonist, an anti-allergic compound, an anti-diabetic compounds, an anti-hyperlipidemic compound, an anti-tussive compound, an angiotensin II antagonist, an angiotensin converting enzyme (ACE) inhibitor, a bronchodilator, an antisense nucleotide, anti-thrombotic and vasodilator compound, an antithrombogenic agent, a phosphodiesterase inhibitor, a tissue plasminogen activator, a thrombolytic agent, a fibrinolytic agent, a vasospasm inhibitor, an endothelin antagonist, an expectorant, an H₂ receptor antagonist, a neural endopeptidase inhibitor, a calcium channel blocker, a potassium channel blocker, a nitrate, a nitric oxide promoter, a vasodilator, an antimicrobial agent, an antibiotic, a platelet reducing agent, a proton pump inhibitor, a rennin inhibitor, a steroid, an anti-mitotic, a microtubule inhibitor, an actin inhibitor, a remodeling inhibitor, an agent for molecular genetic intervention, a cell cycle inhibitor, an inhibitor of the surface glycoprotein receptor, an anti-metabolite, an anti-proliferative agent, a chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, an extracellular matrix component, a cellular component, a biologic agent, ascorbic acid, a free radical scavenger, an iron chelator, an antioxidant, a radiolabelled form or other radiolabelled form of any of the foregoing, or a mixture of any of these.

Exemplary antifungal agents including nystatin, liposomal nystatin, amorolfine, butenafina, naftifine, terbinafine, flucytosine, fluconazole, itraconasole, ketoconazole, posaconazole, ravuconazole, voriconazole, clotrimazole, econasole, miconazole, oxiconazole, sulconazole, terconazole, ticonazole, nikkomycin Z, caspofungin, micafungin, amphotericin B (AmB), AmB lipid complex, AmB colloidal dispersion, pimaricin, griseofulvin, ciclopirox olamine, haloprogin, tolnaftate, undecylrnate.

Exemplary antiviral agents include, but are not limited to, acyclovir, docosanol, ribarivin, interferons, cellulose acetate, carbopol, carrageenan (CAS No. 9000-07-1), pleconaril, amantidine, rimantidine, fomivirsen, zidovudine, lamivudine, zanamivir, oseltamivir, brivudine, abacavir, adefovir, amprenavir, arbidol, atazanavir, atripla, cidofovir, combivir, edoxudine, efavirenz, emtricitabine, enfuvirtide, entecavir, famciclovir, fomivirsen, fosamprenavir, foscarnet, fosfonet, ganciclovir, gardasil, ibacitabine, immunovir, idoxuridine, imiquimod, indinavir, inosine, integrase inhibitor, lamivudine, lopinavir, loviride, mk-0518, maraviroc, moroxydine, nelfinavir, nevirapine, nexavir, nucleoside analogues, oseltamivir, penciclovir, peramivir, pleconaril, podophyllotoxin, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir and zidovudine;

Exemplary anti-microbial agents, include, but are not limited to, acediasulfone, aceturate, acetyl sulfametossipirazine, acetyl sulfamethoxypyrazine, acranil, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, benzoylpas, benzyl penicillin acid, benzyl sulfamide, bicozamycin, bipenam, brodimoprim, capreomycin, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefinenoxime, cefinetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chloramphenicol, chlorotetracycline, cinoxacin, ciprofloxacin, claritromycin, clavulanic acid, clinafloxacin, clindamycin, clofazimine, clofoctal, clometocillin, clomocycline, cloxacillin, cloxyquin, cyclacilline, cycloserine, danoflaxcin, dapsone, deoxycycline, deoxydihydrostreptomycin, dibekacin, dicloxacillin, difloxacin, dihydrostreptomycin, dimetridazole, diminazene, dirirtomycin, doripenam, eflornithine, enoxacin, enrofloxacin, enviomycin, epicillin, erythromycin, etacillin, ethambutol, ethionamide, famciclovir, fenbecillin, fleroxacin, flomoxef, floxacillin, flumequine, furonazide, fortimycin, furazolium chloride, gentamycin, glyconiazide, grepafloxacin, guamecycline, halofuginone, hetacillin, homidium, hydroxyl-stilbamidine, ibostamycin, imidocarb, imipenam, ipronidazole, isoniazide, iosamycin, inosine, lauroguadine, lenampicillin, levofloxin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl) sulfanilanilide, mezlocillin, meziocillin, micronomycin, midecamycin A₁, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin O, penicillin V, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, profiromycin, propamidine, propicillin, protionamide, puraltadone, puromycin, pyrazinamide, pyrimethamine, quinacillin, quinacrine, quinapyramine, quintine, ribostamycin, rifabutine, rifamide, rifampin, rifamycin, rifanpin, rifapentine, rifaxymine, ritipenem, rokitamycin, rolitetracycline, rosamycin, rufloxacin, salazosulfadimidine, salinazid, sancycline, sarafloxacin, sedacamycin, secnidazole, sisomycin, sparfloxacin, spectinomycin, spiramycin, spiramycin I, spiramycin II, spiramycin III, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N⁴-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, tinidazole, tobramycin, tosufloxacin, trimethoprim, troleandromycin, trospectomycin, trovafloxacin, tubercidine, miokamycin, oleandomycin, troleandromycin, vancomycin, verazide, viomycin, virginiamycin and zalcitabine.

Exemplary aldosterone antagonists include, but are not limited to, canrenone, potassium canrenoate, drospirenone, spironolactone, eplerenone (INSPRA®), epoxymexrenone, fadrozole, pregn-4-ene-7,21-dicarboxylic acid, 9,11-epoxy-17-hydroxy-3-oxo, .gamma.-lactone, methyl ester, (7α,11α,17β.)-; pregn-4-ene-7,21-dicarboxylic acid, 9,11-epoxy-17-hydroxy-3-oxo-dimethyl ester, (7α,11α,17β)-; 3′H-cyclopropa(6,7)pregna-4,6-diene-21-carboxylic acid, 9,11-epoxy-6,7-dihydro-17-hydroxy-3-oxo-, .gamma.-lactone, (7α,11α,17β.)-; pregn-4-ene-7,21-dicarboxylic acid, 9,11-epoxy-17-hydroxy-3-oxo-, 7-(1-methylethyl) ester, monopotassium salt, (7α,11α,17β.)-; pregn-4-ene-7,21-dicarboxylic acid, 9,11,-epoxy-17-hydroxy-3-oxo-, 7-methyl ester, monopotassium salt, (7α,11α,17β.)-; 3′H-cyclopropa(6,7) pregna-1,4,6-triene-21-carboxylic acid, 9,11-epoxy-6,7-dihydro-17-hydroxy-3-oxo-, γ-lactone, (6β,7β,11α)-; 3′H-cyclopropa(6,7)pregna-4,6-diene-21-carboxylic acid, 9,11-epoxy-6,7-dihydro-17-hydroxy-3-oxo-, methyl ester, (6β,7β,11α,17β)-; 3′H-cyclopropa (6,7)pregna-4,6-diene-21-carboxylic acid, 9,11-epoxy-6,7-dihydro-17-hydroxy-3-oxo-, monopotassium salt, (6β,7β,11α,17β)-; 3′H-cyclopropa(6,7)pregna-1,4,6-triene-21-carboxylic acid, 9,11-epoxy-6,7-dihydro-17-hydroxy-3-oxo-γ-lactone, (6β,7β,11α,17β)-; pregn-4-ene-7,21-dicarboxylic acid, 9,11-epoxy-17-hydroxy-3-oxo-, .gamma.-lactone, ethyl ester, (7α,11α,17β)-; pregn-4-ene-7,21-dicarboxylic acid, 9,11-epoxy-17-hydroxy-3-oxo-, γ-lactone, 1-methylethyl ester, (7α,11α,17β)-; RU-28318, and the like

Exemplary α-adrenergic receptor antagonists receptor antagonists, include, but are not limited to, phentolamine, tolazoline, idazoxan, deriglidole, RX 821002, BRL 44408, BRL 44409, BAM 1303, labetelol, ifenprodil, rauwolscine, corynathine, raubascine, tetrahydroalstonine, apoyohimbine, akuammigine, .beta.-yohimbine, yohimbol, yohimbine, pseudoyohimbine, epi-3.alpha.-yohimbine, 10-hydroxy-yohimbine, 11-hydroxy-yohimbine, tamsulosin, benoxathian, atipamezole, BE 2254, WB 4101, HU-723, tedisamil, mirtazipine, setiptiline, reboxitine, delequamine, naftopil, saterinone, SL 89.0591, ARC 239, urapidil, 5-methylurapidil, monatepi, haloperidol, indoramin, SB 216469, moxisylyte, trazodone, dapiprozole, efaroxan, Recordati 15/2739, SNAP 1069, SNAP 5089, SNAP 5272, RS 17053, SL 89.0591, KMD 3213, spiperone, AH 11110A, chloroethylclonidine, BMY 7378, niguldipine, and the like.

Exemplary β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hydroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl-, 1-butylamino-3-(2,5-dichlorophenoxy)-2-propanol, 1-isopropylamino-3-(4-(2-cyclopropylmethoxyethyl) phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-165, CP-331684, Fr-172516, ISV-208, L-653328, LM-2616, SB-226552, SR-58894A, SR-59230A, TZC-5665, UK-1745, YM-430, and the like.

Exemplary anti-allergic compounds include, but are not limited to, acrivastine, allociamide, amlexanox, bromexine, cetirizine, clobenzepam, chromoglycate, chromolyn, deslortidine, emedastine, epinastine, fexofenadine, formoterol, hydroxyzine, ketotifen, loratadine, levocabastine, lodoxamide, mabuterol, montelukast, nedocromil, repirinast, salmeterol, seratrodast, suplatast tosylate, terfenadine, tiaramide, and the like.

Exemplary anti-diabetic compounds include, but are not limited to, acarbose, acetohexamide, buformin, carbutamide, chlorpropamide, glibornuride, gliclazide, glimepiride, glipizide, gliquidone, glisoxepid, glyburide, glybuthiazol(e), glybuzole, glyhexamide, glymidine, glypinamide, insulin, metformin, miglitol, nateglinide, phenbutamide, phenformin, pioglitazone, repaglinide, rosiglitazone, tolazamide, tolbutamide, tolcyclamide, troglitazone, voglibose, and the like.

Exemplary anti-hyperlipidemic compounds include, but are not limited to, statins or HMG-CoA reductase inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, pravastatin (PRAVACHOL®), rosuvastatin (CRESTRO®.), simvastatin (ZOCOR®), velostatin (also known as synvinolin), VYTORIN™ (ezetimibe/simvastatin), GR-95030, SQ 33,600, BMY 22089, BMY 22,566, CI 980, and the like; gemfibrozil, cholystyramine, colestipol, niacin, nicotinic acid, bile acid sequestrants, such as, for example, cholestyramine, colesevelam, colestipol, poly(methyl-(3-trimethylaminopropyl) imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like; cholesterol ester transfer protein (CETP) inhibitors, such as for example, CGS 25159, CP-529414 (torcetrapid), JTT-705, substituted N-[3-(1,1,2,2-tetrafluoroethoxy)benzyl]-N-(3-phenoxyphenyl)-trifluoro-3-a-mino-2-propanols, N,N-disubstituted trifluoro-3-amino-2-propanols, PD 140195 (4-phenyl-5-tridecyl-4H-1,2,4-triazole-3-thiol), SC-794, SC-795, SCH 58149, and the like.

Exemplary antitussive compounds, include, but are not limited to, dextromethorphan, carbetapentane, caramiphen, diphenylhydramine, hydrocodene, codeine and the like.

Exemplary angiotensin II antagonists include, but are not limited to, angiotensin, abitesartan, candesartan, candesartan cilexetil, elisartan, embusartan, enoltasosartan, eprosartan, fonsartan, forasartan, glycyllosartan, irbesartan, losartan, olmesartan, milfasartan, medoxomil, ripisartan, pratosartan, saprisartan, saralasin, sarmesin, tasosartan, telmisartan, valsartan, zolasartan, 3-(2′(tetrazole-5-yl)-1,1′-biphen-4-yl)methyl-5,7-dimethyl-2-ethyl-3H-imi-dazo(4,5-b)pyridine, antibodies to angiotensin II, A-81282, A-81988, BAY 106734, BIBR-363, BIBS-39, BIBS-222, BMS-180560, BMS-184698, BMS-346567, CGP-38560A, CGP-42112A, CGP-48369, CGP-49870, CGP-63170, CI-996, CP-148130, CL-329167, CV-11194, DA-2079, DE-3489, DMP-811, DuP-167, DuP-532, DuP-753, E-1477, E-4177, E-4188, EMD-66397, EMD-666R4, EMD-73495, EMD-66684, EXP-063, EXP-929, EXP-3174, EXP-6155, EXP-6803, EXP-7711, EXP-9270, EXP-9954, FK-739, FRI 153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978, L-159282, L-159689, L-159874, L-161177, L-162154, L-162234, L-162441, L-163007, L-163017, LF-70156, LRB-057, LRB-081, LRB-087, LY-235656, LY-266099, LY-285434, LY-301875, LY-302289, LY-315995, ME-3221, MK-954, PD-123177, PD-123319, PD-126055, PD-150304, RG-13647, RWJ-38970, RWJ-46458, S-8307, S-8308, SC-51757, SC-54629, SC-52458, SC-52459, SK 1080, SL-910102, SR-47436, TAK-536, UP-2696, U-96849, U-97018, UK-77778, UP-275-22, WAY-126227, WK-1260, WK-1360, WK-1492, WY 126227, YH-1498, YM-358, YM-31472, X-6803, XH-148, XR-510, ZD-6888, ZD-7155, ZD-8731, ZD 8131, the compounds of ACS registry numbers 124750-92-1, 133240-46-7, 135070-05-2, 139958-16-0, 145160-84-5, 147403-03-0, 153806-29-2, 439904-54-8P, 439904-55-9P, 439904-56-OP, 439904-57-1P, 439904-58-2P, 155918-60-8P, 155918-61-9P, 272438-16-1P, 272446-75-OP, 223926-77-OP, 169281-89-4, 439904-65-1P, 165113-01-9P, 165113-02-OP, 165113-03-1P, 165113-03-2P, 165113-05-3P, 165113-06-4P, 165113-07-5P, 165113-08-6P, 165113-09-7P, 165113-10-OP, 165113-11-1P, 165113-12-2P, 165113-17-7P, 165113-18-8P, 165113-19-9P, 165113-20-2P, 165113-13-3P, 165113-14-4P, 165113-15-5P, 165113-16-6P, 165113-21-3P, 165113-22-4P, 165113-23-5P, 165113-24-6P, 165113-25-7P, 165113-26-8P, 165113-27-9P, 165113-28-OP, 165113-29-1P, 165113-30-4P, 165113-31-5P, 165113-32-6P, 165113-33-7P, 165113-34-8P, 165113-35-9P, 165113-36-OP, 165113-37-1P, 165113-38-2P, 165113-39-3P, 165113-40-6P, 165113-41-7P, 165113-42-8P, 165113-43-9P, 165113-44-OP, 165113-45-1P, 165113-46-2P, 165113-47-3P, 165113-48-4P, 165113-49-5P, 165113-50-8P, 165113-51-9P, 165113-52-OP, 165113-53-1P, 165113-54-2P, 165113-55-3P, 165113-56-4P, 165113-57-5P, 165113-58-6P, 165113-59-7P, 165113-60-OP, 165113-61-1P, 165113-62-2P, 165113-63-3P, 165113-64-4P, 165113-65-5P, 165113-66-6P, 165113-67-7P, 165113-68-8P, 165113-69-9P, 165113-70-2P, 165113-71-3P, 165113-72-4P, 165113-73-5P, 165113-74-6P, 114798-27-5, 114798-28-6, 114798-29-7, 124749-82-2, 114798-28-6, 124749-84-4, 124750-88-5, 124750-91-0, 124750-93-2, 161946-65-2P, 161947-47-3P, 161947-48-4P, 161947-51-9P, 161947-52-OP, 161947-55-3P, 161947-56-4P, 161947-60-OP, 161947-61-1P, 161947-68-8P, 161947-69-9P, 161947-70-2P, 161947-71-3P, 161947-72-4P, 161947-74-6P, 161947-75-7P, 161947-81-5P, 161947-82-6P, 161947-83-7P, 161947-84-8P, 161947-85-9P, 161947-86-OP, 161947-87-1P, 161947-88-2P, 161947-89-3P, 161947-90-6P, 161947-91-7P, 161947-92-8P, 161947-93-9P, 161947-94-OP, 161947-95-1P, 161947-96-2P, 161947-97-3P, 161947-98-4P, 161947-99-5P, 161948-00-1P, 161948-01-2P, 161948-02-3P, 168686-32-6P, 167301-42-OP, 166813-82-7P, 166961-56-4P, 166961-58-6P, 158872-96-9P, 158872-97-OP, 158807-14-8P, 158807-15-9P, 158807-16-OP, 158807-17-1P, 158807-18-2P, 158807-19-3P, 158807-20-6P, 155884-08-5P, 154749-99-2, 167371-59-7P, 244126-99-6P, 177848-35-OP and 141309-82-2P, and the like.

Exemplary angiotensin-converting enzyme inhibitors (ACE inhibitors) include, but are not limited to, alacepril, benazepril (LOTENSIN®, CIBACEN®), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkalnoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like.

Exemplary antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, .beta.-carotene, ubiquinone, ubiquinol-10, tocopherols, coenzyme Q, superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like. Suitable antioxidant enzymes include, but are not limited to, superoxide dismutase, catalase, glutathione peroxidase, NADPH oxidase inhibitors, such as, for example, apocynin, aminoguanidine, ONO 1714, 517834 (benzo(b)pyran-4-one derivative), and the like; xanthine oxidase inhibitors, such as, for example, allopurinol, oxypurinol, amflutizole, diethyldithiocarbamate, 2-styrylchromones, chrysin, luteolin, kaempferol, quercetin, myricetin, isorhamnetin, benzophenones such as 2,2′,4,4′-tetrahydroxybenzophenone, 3,4,5,2′,3′,4′-hexahydroxybenzophenone and 4,4′-dihydroxybenzophenone; benzothiazinone analogues such as 2-amino-4H-1,3-benzothiazine-4-one, 2-guanidino-4H-1,3-benzothiazin-4-one and rhodanine; N-hydroxyguanidine derivative such as, PR5 (1-(3,4-dimethoxy-2-chlorobenzylideneamino-3-hydroxyguanidine); 6-formylpterin, and the like

Exemplary antithrombotic and vasodilator compounds include, but are not limited to, abciximab, acetorphan, acetylsalicylic acid, argatroban, bamethan, benfurodil, benziodarone, betahistine, bisaramil, brovincamine, bufeniode, citicoline, clobenfurol, clopidogrel, cyclandelate, dalteparin, dipyridamol, droprenilamine, enoxaparin, fendiline, ifenprodil, iloprost, indobufen, isobogrel, isoxsuprine, heparin, lamifiban, midrodine, nadroparin, nicotinoyl alcohol, nylidrin, ozagrel, perhexyline, phenylpropanolamine, prenylamine, papaveroline, reviparin sodium salt, ridogrel, suloctidil, tinofedrine, tinzaparin, trifusal, vintoperol, xanthinal niacinate, and the like.

Exemplary bronchodilators include, but are not limited to, ambroxol, atropine, bevonium methyl sulfate, bethanechol, chlorprenaline, cyclodrine, daiphenacine, N-desethyl-oxybutynin, dicyclomine, emepronium, ephedrine, epinephrine, etafredine, ethylnorepinephrine, flavoxate, flutoprium bromide, hexoprenaline, 2-hydroxy-2,2-diphenyl-N-(1,2,3,6-tetra hydro-pyridin-4-ylmethyl)acetamide, ipratropium bromide, isoetharine, NS 21, oxybutynin, oxitropium bromide, propanthelin, propiverine, rispenzepine, terbutaline, 1-teobromine actetic acid, terodiline, tiotropium bromide, tolterodine, trospium, vamicamide, zamiphenacine, and the like.

Exemplary calcium channel blockers include, but are not limited to, amlodipine (NORVASC®), anipamil, aranidipine, aminone, azelnidipine, barnidipine, bencyclane, benidipine, bepridil, cilnidipine, cinnarizine, clentiazem, diltiazem, dotarizine, efonidipine, elgodipine, fantofarone, felodipine, fendiline, flunarizine, fluspirilene, furnidipine, gallopamil, ipenoxazone, isradipine, lacidipine, lemildipine, lercanidipine, lomerizine, manidipine, mibefradil, monatepil, nicardipine, nifedipine, niguldipine, niludipine, nilvadipine, nimodipine, nisoldipine, nitrendipine, nivaldipine, oxodipine, perhexylene, phenyloin, phenylprenylamine, pranidipine, ranolazine, ryosidine, semotiadil, tamolarizine, temiverine hydrochloride, terodiline, tiapamil, vatanidipine hydrochloride, verapamil, ziconotide, AE-0047, CAI, JTV-519, CHF-1521, L-651582, NS-7, NW-1015, RO-2933, SB-237376, SL-34.0829-08, S-312d, SD-3212, TA-993, YM-430, and the like.

Exemplary endothelin antagonists include, but are not limited to, atrasentan, bosentan, darusentan, endothelin, enrasentan, sitaxsentan, sulfonamide endothelin antagonists, tezosentan, BMS 193884, BQ-123, SQ 28608, and the like.

Exemplary expectorants include, but are not limited to, ambroxol, domiodol, erdosteine, guaiacol, guaifenesin, iodinated glycerol, letosteine, mensa, sobrerol, strepronine, terpin, tiopronin, and the like.

Exemplary H₂ receptor antagonists include, but are not limited to, burimamide, cimetidine, ebrotidin, famotidine, nizatidine, roxatidine, rantidine, tiotidine, and the like.

Exemplary neutral endopeptidase inhibitors include, but are not limited to, atrial natriuretic peptides, diazapins, azepinones, ecadotril, fasidotril, fasidotrilat, omapatrilat, sampatrilat, BMS 189,921, Z 13752 A, and the like.

Exemplary NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like.

Exemplary phosphodiesterase inhibitors, include but are not limited to, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI 930, EMD 53998, imazodan, saterinone, loprinone hydrochloride, 3-pyridinecarbonitrile derivatives, acefylline, albifylline, bamifylline, denbufyllene, diphylline, doxofylline, etofylline, torbafylline, theophylline, nanterinone, pentoxofylline, proxyphylline, cilostazol, cilostamide, MS 857, piroximone, milrinone, aminone, tolafentrine, dipyridamole, papaveroline, E4021, thienopyrimidine derivatives, triflusal, ICOS-351, tetrahydropiperazino(1,2-b)beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, tadalafil and vardenafil.

Exemplary potassium channel blockers include, but are not limited to, nicorandil, pinacidil, cromakalim (BRL 34915), aprikalim, bimakalim, emakalim, lemakalim, minoxidil, diazoxide, 9-chloro-7-(2-chlorophenyl)-5H-pyrimido(5,4,-d)(2)-benzazepine, Ribi, CPG-11952, CGS-9896, ZD 6169, diazixide, Bay X 9227, P1075, Bay X 9228, SDZ PCO 400, WAY-120,491, WAY-120,129, Ro 31-6930, SR 44869, BRL 38226, S 0121, SR 46142A, CGP 42500, SR 44994, artilide fumarate, lorazepam, temazepam, rilmazafone, nimetazepam, midazolam, lormetazepam, loprazolam, ibutilide fumarate, haloxazolam, flunitrazepam, estazolam, doxefazepam, clonazepam, cinolazepam, brotizolam, and the like.

Exemplary platelet reducing agents include, but are not limited to, fibrinolytic agents such as for example, ancrod, anistreplase, bisobrin lactate, brinolase, Hageman factor (i.e. factor XII) fragments, plasminogen activators such as, for example, streptokinase, tissue plasminogen activators (TPA), urokinase, pro-urokinase, recombinant TPA, plasmin, plasminogen, and the like; anti-coagulant agents including but are not limited to, inhibitors of factor Xa, factor TFPI, factor VIIa, factor IXc, factor Va, factor VIIIa, inhibitors of other coagulation factors, and the like; vitamin K antagonists, such as, for example, coumarin, coumarin derivatives (e.g., warfarin sodium); glycosoaminoglycans such as, for example, heparins both in unfractionated form and in low molecular weight form; ardeparin sodium, bivalirudin, bromindione, coumarin, dalteparin sodium, danaparoid sodium; dazoxiben hydrochloride, desirudin, dicumarol, efegatran sulfate, enoxaparin sodium, ifetroban, ifetroban sodium, lyapolate sodium, nafamostat mesylate, phenprocoumon, sulfatide, tinzaparin sodium, retaplase; trifenagrel, warfarin, dextrans and the like; abciximab, acadesine, anipamil, argatroban, aspirin, clopidogrel, diadenosine 5′,5′″-P1,P4-tetraphosphate (Ap4A) analogs, difibrotide, dilazep dihydrochloride, dipyridamole, dopamine, 3-methoxytyramine, glucagon, glycoprotein IIb/IIIa antagonists, such as, for example, Ro-43-8857, L-700,462, iloprost, isocarbacyclin methyl ester, itazigrel, ketanserin, BM-13.177, lamifiban, lifarizine, molsidomine, nifedipine, oxagrelate, prostaglandins, platelet activating factor antagonists such as, for example, lexipafant, prostacyclins, pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612, ZK-36374,2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as, for example, picotamide, sulotroban, ticlopidine, tirofiban, trapidil, ticlopidine, trifenagrel, trilinolein, 3-substituted 5,6-bis(4-methoxyphenyl)-1,2,4-triazines; antibodies to glycoprotein IIb/IIIa; anti-serotonin drugs, such as, for example, clopridogrel; sulfinpyrazone and the like; aspirin; dipyridamole; clofibrate; pyridinol carbamate; glucagon, caffeine; theophyllin pentoxifyllin; ticlopidine, and the like.

Exemplary proton pump inhibitors include, but are not limited to, disulprazole, esomeprazole, lansoprazole, leminoprazole, omeprazole, pantoprazole, rabeprazole, timoprazole, tenatoprazole, 2-(2-benzimidazolyl)-pyridine, tricyclic imidazole, thienopydidine benzimidazole, fluoroalkoxy substituted benzimidazole, dialkoxy benzimidazole, N-substituted 2-(pyridylalkenesulfinyl)benzimidazole, cycloheptenepyridine, 5-pyrrolyl-2-pyridylmethylsulfinyl benzimidazole, alkylsulfinyl benzimidazole, fluoro-pyridylmethylsulfinyl benzimidazole, imidazo(4,5-b)pydridine, RO 18-5362, IY 81149, 4-amino-3-carbonyl quinoline, 4-amino-3-acylnaphthyride, 4-aminoquinoline, 4-amino-3-acylquinoline, 3-butyryl-4-(2-methylphenylamino)-8-(2-hydroxyethoxy)quinoline, quinazoline, tetrahydroisoquinolin-2-yl pyrimidine, YH 1885, 3-substituted 1,2,4-thiadiazolo(4,5-a)benzimidazole, 3-substituted imidazo(1,2-d)-thiadiazole, 2-sulfinylnicotinamide, pyridylsulfinylbenz imidazole, pyridylsulfinyl thieno imidazole, theinoimidazole-toluidine, 4,5-dihydrooxazole, thienoimidazole-toluidine, Hoe-731, imidazo(1,2-a)pyridine, pyrrolo(2,3-b)pyridine, and the like.

Exemplary renin inhibitors include, but are not limited to, aldosterone, aliskiren (SPP-100), ditekiren, enalkrein (A-64662), medullipin, terlkiren, tonin, zankiren, RO 42-5892 (remikiren), A 62198, A 64662, A 65317, A 69729, A 72517 (zankiren), A 74273, CP 80794, CGP 29287, CGP-38560A, EMD 47942, ES 305, ES 1005, ES 8891, FK 906, FK 744, H 113, H-142, KRI 1314, pepstatin A, RO 44-9375 (ciprokiren), RO 42-5892, RO 66-1132, RO 66-1168, SP 500, SP 800, SR-43845, SQ 34017, U 71038, YM-21095, YM-26365, urea derivatives of peptides, amino acids connected by nonpeptide bonds, di- and tri-peptide derivatives (e.g., Act-A, Act-B, Act-C, ACT-D, and the like), amino acids and derivatives thereof, diol sulfonamides and sulfinyls, modified peptides, peptidyl beta-aminoacyl aminodiol carbamates, monoclonal antibodies to renin.

Exemplary COX-2 inhibitors include, but are not limited to, nimesulide, celecoxib (CELEBREX®), etoricoxib (ARCOXIA®), flosulide, lumiracoxib (PREXIG®, COX-189), parecoxib (DYNSTAT®), rofecoxib (VIOXX®), tiracoxib (JTE-522), valdecoxib (BEXTRA®), ABT 963, BMS 347070, CS 502, DuP 697, GW-406381, NS-386, SC-57666, SC-58125, SC-58635, and the like, and combinations of two or more thereof.

Exemplary steroids include, but are not limited to, 21-acetoxypregnenolone, alcolometasone, algestone, amcinonide, beclomethasone, betamethasone, budesonide, chlorprednisone, clobetasol, clobentasone, clocortolone, cloprednol, corticosterone, cortisine, corticazol (cortivatol), deflazacort, desonide, desoximetasone, dexamethasone, diflorasone, diflucortolone, difluprednate, enoxolone, fluzacort, flucloronide, flumethasone, flunisolide, flucinolone acetonide, fluocininide, fluocortin butyl, fluocortolone, fluorometholone, fluperolone acetate, fluprednidene acetate, fluprednisolone, flurandrenolide, fluticasone propionate, fluticasone propionate, formocortal, halcinonide, halobetasol propionate, halometasone, haloprednone acetate, hydrocortamate, hydrocortisone and its derivatives (such as phosphate, 21-sodium succinate and the like), hydrocortisone terbutate, isoflupredone, loteprednol etabonate, mazipredone, medrysone, meprednisone, methylprednisolone, mometasone furoate, paremethasone, prednicarbate, prednisolone and its derivatives (such as 21-stearoylglycolate, sodium phosphate and the like), prednisone, prednival, prednylidene and its derivatives (such as 21-diethylaminoactetate and the like), rimexolone, tixocortol, trimcinolone and its derivatives (such as acetonide, benetonide and the like), and the like.

The combination therapies described herein is provided in a combined amount effective to inhibit QS in the bacteria (and/or treat a bacterial infection associated with bacterial QS and/or treat a disorder associated with biofilm formation). This process may involve administering to a subject in need thereof one or more ellagitannins and a standard of care anti-bacterial therapeutic (and/or additional therapeutic/second agent) at the same time, which may be achieved by administering a single composition or pharmacological formulation that includes both an ellagitannin and a standard of care therapeutic, or by administering two distinct compositions or formulations, at the same time, wherein one composition includes an ellagitannin and the other includes a standard of care anti-bacterial therapeutic. In another embodiment, the combination therapy involves administering to a subject in need thereof an ellagitannin and a standard of care anti-bacterial therapeutic (and/or additional therapeutic/second agent) at different times, which may be achieved by administering two distinct compositions or formulations, at different time intervals, wherein one composition includes an ellagitannin and the other includes a standard of care anti-bacterial therapeutic (and/or additional therapeutic/second agent).

Alternatively, the treatment with the ellagitannin(s) may precede or follow the treatment with the standard of care anti-bacterial therapeutic (and/or additional therapeutic/second agent) by intervals ranging from minutes to weeks. In embodiments where the ellagitannin(s) and the standard of care anti-bacterial therapeutic (and/or additional therapeutic/second agent) are administered separately (either in separate compositions administered simultaneously or in separate compositions administered at different time intervals), one would generally ensure that a significant period of time did not expire between the times of each delivery, such that the further therapeutic agent and the ellagitannin would still be able to exert an advantageously combined effect. In such instances, it is contemplated that one would administer both modalities within about 1, about 2, about 3, about 4, about 5, about 5, about 6, about 7, about 8, about 9, about 10, about 11, about 12, about 13, about 14, about 15, about 16, about 17, about 18, about 19, about 20, about 21, about 22, about 23, about 24, about 36, about 48, or about 72 hours of each other. In one embodiment, both modalities are administered within about 6-12 hours of each other. In some situations, it may be desirable to extend the time period for treatment significantly. Exemplary routes of administration of the peptides or compositions described herein include, but are not limited to, intradermal, intramuscular, intraperitoneal, intraocular, intravenous, subcutaneous, topical, oral and intranasal administration.

C. Medical Devices

In another embodiment, one or more ellagitannins is used to inhibit biofilm formation associated with bacterial QS on a medical device by contacting the device with an ellagitannin in an amount effective to inhibit biofilm formation. Percutaneous devices (such as catheters) and implanted medical devices (including, but not limited to, pacemakers, vascular grafts, stents, and heart valves) commonly serve as foci for bacterial infection. The tendency of some microorganisms to adhere to and colonize the surface of the device promotes such infections, which increase the morbidity and mortality associated with use of the devices.

For example, one or more ellagitannins is used to inhibit biofilm formation on substrates used to manufacture medical devices associated with non-invasive and invasive medical procedures. Such substrates include, without limitation, tubular, sheet, rod and articles of proper shape for use in a number of medical devices such as vascular grafts, aortic grafts, arterial, venous, or vascular tubing, vascular stents, dialysis membranes, tubing or connectors, blood oxygenator tubing or membranes, surgical instruments, ultrafiltration membranes, intra-aortic balloons, stents, blood bags, catheters, sutures, soft or hard tissue prostheses, synthetic prostheses, prosthetic heart valves, tissue adhesives, cardiac pacemaker leads, artificial organs, endotracheal tubes, lenses for the eye such as contact or intraocular lenses, blood handling equipment, apheresis equipment, diagnostic and monitoring catheters and sensors, biosensors, dental devices, drug delivery systems, or bodily implants of any kind. For example, arthroscopic surgery is routinely performed with use of medical devices that minimize the invasiveness of the procedure. Such devices include, for example and without limitation, ultrathin microfiberoptic endoscopes that offer the laryngologist unique access to the limited spaces of the temporal bone and skull base. In another example, a stent supplemented with one or more ellagitannins can be constructed. Stents are used to maintain an open lumen in tissues including the tracheo-bronchial system, the biliary hepatic system, the esophageal bowel system, and the urinary tract system.

III. Routes of Administration and Dosage

Ellagitannin(s) either alone or in combination with a standard of care anti-bacterial therapeutic as described herein are administered by any route that delivers an effective dosage to the desired site of action, with acceptable (preferably minimal) side-effects. Numerous routes of administration are known, including for example, oral, rectal, vaginal, transmucosal, buccal or intestinal administration; parenteral delivery, including intraperitoneal, intramuscular, subcutaneous, intramedullary injections, as well as intrathecal, cutaneous or intradermal injections; respiratory or inhalation, nasal, pulmonary and topical application, including ocular and transdermal applications.

When used in the above or other treatments, a “therapeutically-effective amount” or an “effective amount” of an ellagitannin or a composition comprising an ellagitannin means a sufficient amount of the ellagitannin is provided to treat disorders or to achieve a desired result. It will be understood, however, that the total daily usage of the ellagitannin in a therapeutic method described herein will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The dose of ellagitannin administered to a mammalian subject range from about 10 μg to about 400 mg/day. In some embodiments, the dose is about 10 μg/day, about 25 μg/day, about 50 μg/day, about 75 μg/day, about 100 μg/day, about 125 μg/day, about 150 μg/day, about 175 μg/day, about 200 μg/day, about 225 μg/day, about 250 μg/day, about 275 μg/day, about 300 μg/day, about 325 μg/day, about 350 μg/day, about 375 μg/day, about 400 μg/day, about 425 μg/day, about 450 μg/day, about 475 μg/day, about 500 μg/day, about 750 μg/day, about 1 mg/day, about 5 mg/day, about 10 mg/day, about 25 mg/day, about 30 mg/day, about 40 mg/day, about 45 mg/day, about 50 mg/day, about 60 mg/day, about 70 mg/day, about 80 mg/day, about 90 mg/day, about 100 mg/day, about 150 mg/day, about 200 mg/day, about 250 mg/day, about 300 mg/day, about 350 mg/day or about 400 mg/day. In some embodiments, the maximum dosage is about 200 mg/day. In some embodiments, the maximum dosage is about 300 mg/day. If desired, the effective daily dose is divided into multiple doses for purposes of administration; consequently, single dose compositions may contain such amounts or submultiples thereof to make up the daily dose. The dosage regimen of an ellagitannin composition alone or in combination as described herein to be used in treatment of bacterial infections (or biofilm formation) associated with QS will be determined by the attending physician considering various factors which modify the action of the ellagitannin, e.g., the patient's age, sex, and diet, the severity of any infection, time of administration and other clinical factors.

Oral dosage forms include tablets, capsules, caplets, solutions, suspensions and/or syrups, and may also comprise a plurality of granules, beads, powders or pellets that may or may not be encapsulated. Such dosage forms are prepared using conventional methods known to those in the field of pharmaceutical formulation and described in the pertinent texts, e.g., in Remington: The Science and Practice of Pharmacy, supra). Tablets and capsules represent the most convenient oral dosage forms, in which case solid pharmaceutical carriers are employed.

Tablets include those manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets are also molded rather than compressed, starting with a moist or otherwise tractable material.

In addition to the ellagitannin either alone or in combination as described herein, tablets prepared for oral administration will in one aspect contain other materials such as binders, diluents, lubricants, disintegrants, fillers, stabilizers, surfactants, preservatives, coloring agents, flavoring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form also includes a capsule, in which case the ellagitannin-containing composition is in one aspect encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals.

Solid dosage forms, whether tablets, capsules, caplets, or particulates, are, if desired, coated so as to provide for delayed release. Dosage forms with delayed release coatings are in one aspect manufactured using standard coating procedures and equipment. Such procedures are known to those skilled in the art and described in the pertinent texts (See, for e.g., Remington: The Science and Practice of Pharmacy, supra). In one aspect, after preparation of the solid dosage form, a delayed release coating composition is applied using a coating pan, an airless spray technique, fluidized bed coating equipment, or the like. Delayed release coating compositions comprise in various aspects a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and optionally are delayed release. As will be appreciated by those of ordinary skill in the art, sustained release dosage forms are formulated in various aspects by dispersing a drug within a matrix of a gradually bioerodible (hydrolyzable) material such as, for example, an insoluble plastic, a hydrophilic polymer, or a fatty compound, or by coating a solid, drug-containing dosage form with such a material. Insoluble plastic matrices are in certain aspects comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. and without limitation copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Although compositions described herein are in one aspect administered orally, other modes of administration are contemplated as well. Exemplary modes of administration include transmucosal (e.g., U.S. Pat. Nos. 5,288,498; 6,248,760; 6,355,248; 6,548,490, the disclosures of which are incorporated herein by reference in their entireties), transurethral (e.g., e.g., U.S. Pat. Nos. 5,919,474 and 5,925,629, the disclosures of which are incorporated herein by reference in their entireties), vaginal or perivaginal (e.g., U.S. Pat. Nos. 4,211,679; 5,491,171 and 6,576,250, the disclosures of which are incorporated herein by reference in their entireties) and intranasal or inhalation (e.g., U.S. Pat. Nos. 4,800,878; 5,112,804; 5,179,079; 6,017,963; 6,391,318 and 6,815,424, the disclosures of which are incorporated herein by reference in their entireties). One of skill in the art would be able to modify a composition comprising an ellagitannin either alone or in combination with a standard of care anti-bacterial therapeutic as described herein to be used in any of the modes of administration described herein.

Compositions comprising an ellagitannin either alone or in combination as described herein are be used as a topical agent. The topical agent is a solution, that is, in one aspect, a liquid formulation comprising the ellagitannin and a carrier. Other suitable forms include semi-solid or solid forms comprising a carrier indigenous to topical application and having a dynamic viscosity preferably greater than that of water, provided that the carrier does not deleteriously react with the ellagitannin in the composition. Suitable formulations include, but are not limited to, lip balms, suspensions, emulsions, creams, ointments, powders, liniments, salves and the like. If desired, these compositions may be sterilized or mixed with auxiliary agents, including but not limited to, preservatives, stabilizers, wetting agents, buffers or salts for influencing osmotic pressure and the like well known in the art. Preferred vehicles for semi-solid or solid forms topical preparations include ointment bases, conventional ophthalmic vehicles; creams; and gels. These topical preparations optionally contain emollients, perfumes, and/or pigments to enhance their acceptability for various usages, provided that the additives do not deleteriously react with the ellagitannin material in the composition.

Also suitable for topical application are sprayable aerosol preparations wherein the ellagitannin, preferably in combination with a solid or liquid inert carrier material, is packaged in a squeeze bottle or in admixture with a pressurized volatile, normally gaseous propellant, e.g., a Freon (chlorofluorocarbon) or environmentally acceptable volatile propellant. Such compositions are used for in one aspect, application to environmental surfaces, e.g., examining tables, toilet seats and the like, and/or for application to the skin or to mucous membranes. The aerosol or spray preparations optionally contain solvents, buffers, surfactants, perfumes, and/or antioxidants in addition to the ellagitannin.

The compositions are in certain aspects employed in mixture with conventional excipients, i.e., pharmaceutically acceptable organic or inorganic carrier substances, suitable for topical application which do not deleteriously react with the ellagitannin in the composition. The compositions of the invention optionally include diluents, fillers, salts, buffers, stabilizers, solubilizers, and other materials well known in the art (Remington's Pharmaceutical Sciences 16th edition, Osol, A. Ed. (1980).

IV. Kits and Unit Doses

In related variations of the preceding embodiments, a composition comprising an ellagitannin as described herein packaged alone, e.g., in a kit or package or unit dose, or is optionally arranged to permit co-administration with one or more other therapeutic agents as described herein, but the ellagitannin and the agent are not in admixture. In an alternative variation, the ellagitannin and the agent are in admixture. In some embodiments, the two components to the kit/unit dose are packaged with instructions for administering the two agents to a human subject for treatment of one of the above-indicated disorders and diseases. The kit may comprise a composition described herein in combination with a vehicle in a cream or gel base, as a pump-spray, as an aerosol, on an impregnated bandage, or in a dropper.

### EXAMPLES

### Example 1

### Ellagitannins Inhibited Bacterial QS

The present Example describes the isolation and verification of two C-glycosidic ellagitannins, castalagin and vescalagin, from C. erectus and the confirmation of anti-QS activity of these compounds.

Materials and Methods:

Plant Extraction:

Leaves of the medicinal plant C. erectus (Combretaceae), were collected and processed according to methods described previously (Adonizio et al., 2006). Briefly, pulverized plant material was extracted into boiling water, freeze-dried using a lyophilizer, and stored at −20° C. until needed.

Bioassay-Guided Fractionation:

Anti-QS activity was confirmed in the crude extract (Adonizio et al., 2006) and followed throughout the separation process using the P. aeruginosa PAO1-derived biomonitor strains pPCS 1001 and pPCS 1002 (Pesci et al., 1997). These strains harbor lacZ fusions to the QS gene promoter regions enabling blue/white selection for QS activity. Bioassays were carried out as previously described (Adonizio et al., 2006) with some modification. Briefly, LB agar plates were seeded with a lawn of one of the biomonitor strains and allowed to dry for 1 hour. Small wells were then cut and aspirated from the agar, and 10 μl aliquots of each fraction were pipetted into each well. Wells were checked at 18 h and 24 h for zones of QS inhibition.

Preliminary Thin Layer Chromatography:

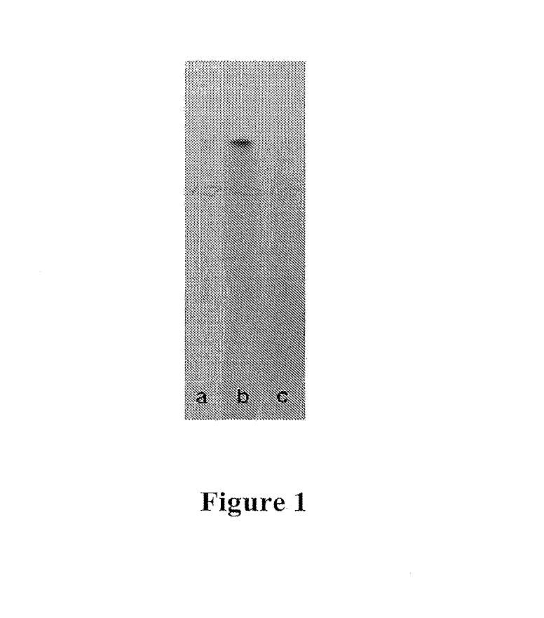

Approximately 1 μl of crude extract was spotted to reverse phase thin layer chromatography (TLC) plates (two spots run in tandem). Adequate separation for visualization of three distinct bands from each spot was achieved with an 80:20 acetonitrile/H₂0 mobile phase containing 0.1% formic acid (FIG. 1, lane A). A 1% ferric chloride stain was applied to one-half of the plate as a phenolic indicator (FIG. 1, lane B). The other side was overlaid with agar containing one of the aforementioned PAO1 biomonitor strains to indicate anti-QS activity (FIG. 1, lane C).

Fractionation Methods:

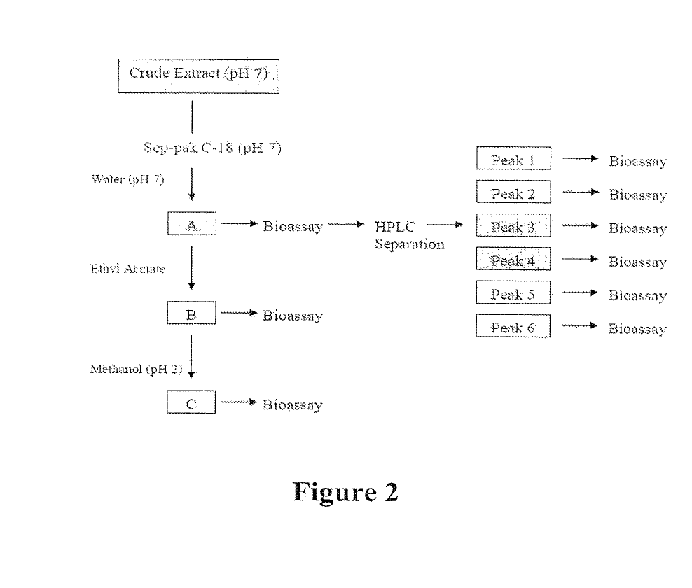

The fractionation of crude aqueous extract prior to HPLC separation is illustrated in FIG. 2. Separation was patterned after methods developed for wine polyphenolics (Sun et al., 2006). A CI8 PrepSep column (Fisher Scientific 11-131-11 5 g/20 ml) was conditioned with 200 ml methanol followed by 200 ml water at pH 7. The flow rate of approximately 2 ml/min was controlled through positive pressure applied via syringe. The crude aqueous extract of C. erectus (0.25 g) was resuspended in 5 ml water, adjusted to pH 7 with sodium hydroxide, and added to the column. Fraction A was eluted with approximately 150 ml water at pH 7. The column was then washed with 100 ml water and dried under vacuum for several seconds. Fraction B was then eluted with approximately 400 ml ethyl acetate. The column was washed with ethyl acetate and dried under vacuum before elution of fraction C with 200 ml of acidified methanol. The presence of phenolics in each fraction was monitored by periodically spotting to TLC plates coated with ferric chloride reagent. Each fraction was evaporated to dryness before bioassay at 1 mg/ml concentration.

HPLC Separation:

Fraction A was separated on an Agilent 1200 series LC system (Agilent Technologies, USA) using an Altima C18 column (51.i, 10×250 mm; 1004 injection volume). A water-acetonitrile mobile phase with 0.1% formic acid was used with a flowrate of 1.5 ml/min. Conditions were as follows: 0-2 min, 0% acetonitrile; 2-47 min, 0-40% acetonitrile; 47-48 min, 40-100% acetonitrile; 48-50 min, 100% acetonitrile; 50-51 min, 100-0% acetonitrile. Fractions were collected manually based on absorbance at 313 nm and tested for anti-QS activity.

Mass Spectrometric Analysis:

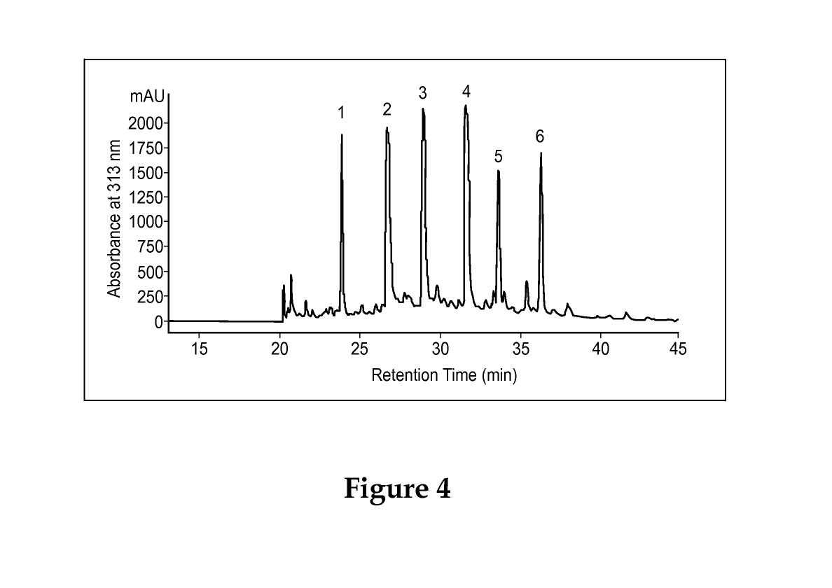

Direct injection electrospray ionization mass spectroscopy and MS-MS analysis (Esquire 3000+, Ion Trap Mass Spectrometer Bruker Daltonics, Germany) were used for mass identification of vescalagin and castalagin. The isolated peaks 3 and 4 (FIG. 4) were injected directly into the ESI source with a syringe pump at a flow rate of 0.2 ml/min. Nebulizer gas was maintained at 7 psi. Capillary temperature was set at 300° C. with a voltage of 3.5 kV. Spectra were obtained in negative ion mode with a scanning range of 100-1000 m/z. MS-MS of compounds were also acquired in negative ion mode. However, the conditions were modified by increasing capillary temperature and voltage to 325° C. and 4 kV respectively. Trap rolling and smart fragmentation settings were activated, and the instrument was set to scan from 50-1000 m/z. Exact mass measurements were made with a Waters Q-Tof2 using reserpine as a lock mass. Samples were introduced via LC flow and reserpine flow from a syringe pump was T-ed in. Spectra were obtained in the positive ion mode with a scanning range from 100-1000 m/z.

NMR Spectroscopy:

NMR spectra were recorded using a Varian Inova 600 M1-[z FT-NMR spectrometer with D20 acidified with d-TFA or D20:[D6]-acetone (8:2) as solvents. Proton spectra were obtained using standard parameters. The structure was elucidated using COSY, HMQC, and HMBC, and through comparison with the standard spectra of vescalagin and castalagin.

Biological Assays:

Assays for AHL production, QS gene activity, and virulence factor production (LasA, LasB, and pyoverdin) were carried out as detailed in our previous work (Adonizio et al., 2008b). Samples were tested at the following concentrations: 1 mg/ml crude extract of C. erectus, 40 μg/ml crude extract, 40 μg/ml vescalagin, or 40 μg/ml castalagin. These additions were compared to a media-only control for the reduction of QS. Prototypic P. aeruginosa strain PAO1 (Holloway & Morgan, 1986), and its promoter-fusion derivatives P_{lasR}-lacZ (pPCS1001), and P_{rhlR}-lacZ (pPCS1002), (Adonizio et al., 2008b; Pesci et al., 1997) were used throughout this study. In addition, Staphylococcus aureus (ATCC #12600) was used in the LasA staphylolytic assay.

Results

The data presented herein demonstrate the isolation and verification of two C-glycosidic ellagitannins, castalagin and vescalagin, from C. erectus and the confirmation of anti-QS activity of these compounds. The isolation procedure was largely directed by anti-QS bioassays using P. aeruginosa strains containing a lacZ fusion to the QS gene lasR or rh1R (Adonizio et al., 2008b; Pesci et al., 1997). Each fraction was pipetted into a small well in an agar plate seeded with one of these biomonitor strains and the appropriate reagents for visualization of lacZ activity. Active fractions resulted in a change of color in the biomonitor strain from blue to off-white in the area surrounding the well indicating anti-QS activity. Only fractions with activity were subjected to further separation.

TLC Reveals a Phenolic Compound Responsible for Anti-QS Activity.

Prior to column chromatography, separation was attempted with various mobile phases on thin layer chromatography (TLC) plates. Reverse phase TLC of crude extract of C. erectus using an acidified acetonitrile/water mobile phase revealed two long-wave UV-reactive bands and one chromatic band, the latter being brown in color and slightly tailing (FIG. 1A).

The plates were reacted with a number of different agents, the most notable being ferric chloride, a stain for phenolic compounds. Ferric chloride staining resulted in the chromatic band turning dark blue, indicating phenolic compounds in this region (FIG. 1B). An unstained portion of the TLC plate was overlaid with agar containing an anti-QS biomonitor strain derived from P. aeruginosa (PlasR-lacZ (pPCS1001)) (Adonizio et al., 2008b; Pesci et al., 1997). Anti-QS activity was visualized as a loss of blue color over the phenolic band due to reduced lasR expression and linked β-galactosidase activity (FIG. 1C).

Anti-QS activity against the tested lasR biomonitor strain correlates with our previous data on the reduction of lasR gene expression seen with the crude extract of C. erectus (Adonizio et al., 2008b). The localization of this activity indicates phenolic compounds are responsible for the anti-QS activity seen in this species. Phenolic compounds have been previously shown to interfere with bacterial QS (Huber et al., 2004).

Fractionation of Polyphenolics in Crude Extract.

Since the TLC staining and overlay procedure revealed the phenolic band to contain the anti-QS activity in C. erectus, larger-scale fractionation was then tailored to separation of these compounds. We adopted a method based on the resolution of wine polyphenols (Sun et al., 2006). A schematic of fractionation can be seen in FIG. 2.

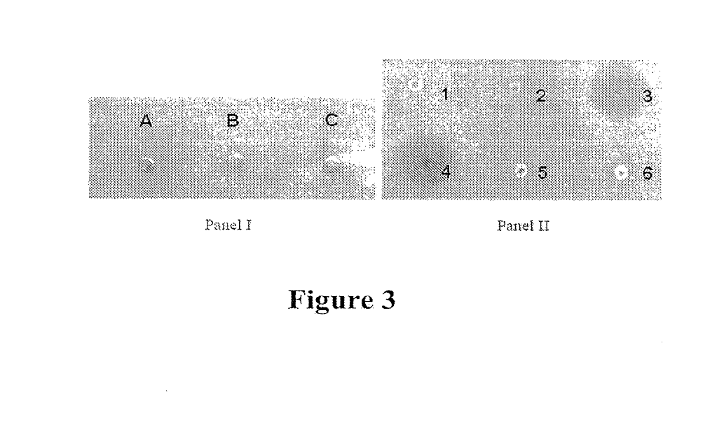

Crude aqueous extract of C. erectus was separated into three fractions based on solvent polarity. Fraction A eluted with water as a bright yellow liquid believed to contain phenolic acids and hydrolyzable tannins according to prior work on polyphenolic separation (Oszmianski et al., 1988; Sun et al., 2006). Fraction B, which eluted with ethyl acetate, was colorless to pale yellow and likely contained colorless proanthocyanins, flavanols, and some monomer and oligomer phenolic acids (Oszmianski et al., 1988; Sun et al., 2006). Fraction C, which eluted with acidified methanol, was dark brown indicating the presence of complex tannins, pigmented proanthocyanidins, and pyranoanthocyanins (Oszmianski et al., 1988; Sun et al., 2006). The presence of phenolics in each fraction was monitored by periodically spotting to TLC plates coated with ferric chloride reagent. Each fraction was tested for anti-QS effect revealing Fraction A to contain the majority of activity (FIG. 3, (Panel I)).

Fraction A is the most polar fraction and thus contains phenolic acids and hydrolyzable tannins. Prior work on ellagic acid and EGCG (a hydrolyzable tannin) indicated that these compounds outperformed complex tannins (such as those found in Fraction C) in the inhibition of QS (Huber et al., 2004).

HPLC Separation Revealed Two Fractions with Anti-QS Activity.

The separation of fraction A via HPLC resulted in six major peaks designated 1 to 6, and a number of minor peaks (FIG. 4). The detection wavelength was set at 313 nm based on work by Oszmianski et al (Oszmianski et al., 1988). Fractions 1 through 6 eluted at approximately 24, 26.8, 29, 31.7, 33.8, and 36.4 minutes, respectively. Each fraction was collected and tested with the anti-QS biomonitor strains revealing activity in Fractions 3 and 4 (FIG. 3, (Panel II)).

Vescalagin and Castalagin Elucidated as Active Compounds.

Fractions 3 and 4 were checked for purity by TLC and behaved as pure compounds (single, non-tailing bands). Both Fractions 3 and 4 when subjected to mass spectrometric analysis produced a strong peak at m/z 933 μM-HI and a smaller fragment peak at m/2z 466 [M-H]2

MS-MS of compounds 3 and 4 revealed strong peaks at m/z 915 and 613. The former being simply the parent compound minus water, and the latter indicating the loss of ellagic acid (302 daltons), a fragment regarded as diagnostic of ellagitannins (Tang & Hancock, 1995). A literature review suggested that these compounds may be the ellagitannins vescalagin and castalagin (Okuda et al., 1993; Tang & Hancock, 1995) as they both have a molecular weight of 934 and contain ellagic acid components. Exact mass measurements revealed a mass of 935.0811 [M+1] for Fraction 3 and 935.0794 for Fraction 4 [M+1], a difference of 2.8 and 1.0 ppm, respectively. The calculated exact mass for the [M+1] ion of both vescalagin and castalagin is: 935.0785 for C₄₁H₂₇O₂₆⁺.

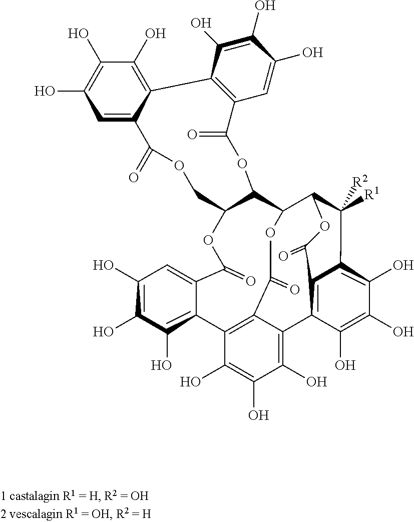

NMR data were compared with spectra from the known compounds (Glabasnia & Hofmann, J. Agric. Food Chem., 54:3380-3390, 2006; Tang & Hancock, 1995), thus confirming the identity of Fractions 3 and 4 as vescalagin and castalagin, respectively (FIG. 5). To avoid confusion, these fractions will hereafter be referred to by their compound names.

Bioassays on P. Aeruginosa Confirm Anti-QS Activity of Ellagitannins.

The anti-QS activity of the ellagitannins vescalagin and castalagin is corroborated by prior work on the QS-inhibiting properties of ellagic acid (Huber et al., 2004). However, specific bioassays were necessary to elaborate the precise effect on P. aeruginosa QS and virulence. Here, we compare the effect of the purified compounds to that of the crude extract of C. erectus examined in our previous works (Adonizio et al., 2008a).

Polyphenolics have been shown to act as QS-inhibitors at a concentration range of 20-60 μg/ml (Huber et al., 2004) thus a 40 μg/ml concentration was chosen for the purified ellagitannins. For comparison, we tested 40 mg/ml and 1 μg/ml concentrations of crude extract, the latter being the working concentration for our previous studies on of C. erectus (Adonizto et al., 2008a; Adonizio et al., 2008b).

LasA Protease Activity is Reduced in the Presence of Ellagitannins.

LasA belongs to the p-lytic endopeptidase class of proteases (Kessler, 1995) and plays a major role in host tissue degradation (Kharazmi, 1989; Morihara & Homma, 1985). LasA protease activity was determined by measuring the ability of culture supernatants to lyse boiled S. aureus cells (Kong et al., Int. J. Med. Microbiol., 296:133-139, 2006). There was a significant decrease in LasA activity compared to that of the control when strain PAO1 was grown in the presence of C. erectus crude extract at 1 mg/ml (91% decrease), and a lesser effect at 40 pg/ml (26% decrease) (Table 1). Purified vescalagin and castalagin affected significant reductions in LasA activity as well, with decreases of 73% and 80%, respectively (Table 1). The significant effect on LasA protease production is in agreement with our previous data on C. erectus (Adonizio et al., 2008b), and suggests that vescalagin and castalagin are responsible for the reduction in LasA activity.

LasB Elastase Activity is Reduced in the Presence of Ellagitannins.

LasB elastase is a zinc metalloprotease capable of affecting the host immune system and destroying biological tissue (Bever & Iglewski, 1988). The elastolytic activity of culture supernatants was determined using elastin Congo red (ECR; Sigma, St. Louis, Mo.) (Ohman et al., 1980). There was a significant decrease in LasB activity compared to that of the control when PAO1 was grown in the presence of C. erectus at 1 mg/ml (70% decrease) or 40 ug/ml (60% decrease) (Table 1). Purified vescalagin and castalagin also affected significant reductions in LasB activity, with decreases of 67% and 63%, respectively (Table 1). The effect on LasB production agrees with our previous data (Adonizio et al., 2008b) on C. erectus, and suggests vescalagin and castalagin are responsible for the reduction in LasB activity.

TABLE 1
Effect of crude extract of C. erectus and purified ellagitannins
on P. aeruginosa virulence factors.

| | | | Pyoverdin |
|---|---|---|---|
| Culture Conditions | LasA Activity^{a} | Elastase Activity^{b} | production^{c} |
| Media only | 0.243 ± 0.024 | 142.7 ± 17.7 | 4710 ± 238 |
| Crude 1 mg/ml | 0.021 ± 0.004 | 42.9 ± 2.1 | 700 ± 212 |
| Crude 40 μg/ml | 0.179 ± 0.013 | 56.7 ± 5.5 | 4208 ± 205 |
| Vescalagin | 0.066 ± 0.008 | 47.2 ± 6.8 | 4226 ± 138 |
| Castalagin | 0.048 ± 0.003 | 52.7 ± 9.0 | 4207 ± 150 |

^{a}LasA activity was expressed as reduction in OD₆₀₀/hour per μg total protein.
^{b}Elastase activity was expressed as absorbance at OD₄₉₅ per μg of protein × 1000.
^{c}Pyoverdin production expressed as fluorescence (405/465 nm) per μg of protein.

Previous studies indicate a 40% decrease in LasB activity when the related organism Pseudomonas putida is grown in the presence of 30 μg/ml ellagic acid (a component of ellagitannins) (Huber et al., 2004).

Ellagitannins do not Affect Pyoverdin Production.

Pyoverdins function as siderophores essentially starving host tissues by competing with mammalian transferrin for iron. They also promote pathogenicity by stimulating bacterial growth (Cox & Adams, 1985), while auto-regulating themselves and the production of other toxins. Relative concentration of pyoverdin was based on fluorescence of the supernatant at an excitation wavelength of 405 nm and an emission wavelength of 465 nm (Gemini EM microplate reader). None of the experimental conditions significantly affected pyoverdin production, with the exception of C. erectus at a concentration of 1 mg/ml (85% reduction) (Table 1). This is consistent with our previous data on C. erectus (Adonizio et al., 2008b).

The inability of the purified compounds to reduce pyoverdin levels suggests that the crude extract contains some factor other than the ellagitannins that is responsible for this effect. No significant reduction occurred with a crude extract concentration of 40 μg/ml, indicating a relatively high amount of this unknown factor is needed for an effect on pyoverdin levels.

Ellagitannins Affect the Production of QS Signaling Molecules.

P. aeruginosa manufactures two main QS signaling molecules N-(3-oxododecanoyl)-L-homoserine lactone (OdDHL) and N-butanoyl-L-homoserine lactone (BHL) called autoinducers (Pearson et al., Proc. Natl. Acad. Sci. USA, 92:1490-1494, 1994). These molecules diffuse into the environment, and when they reach a putative threshold concentration, they activate the QS receptor genes. Inhibition of these signals has been shown to cause attenuation of pathogenicity (Adonizio et al., 2008a; Adonizio et al., 2008b; Manefield et al., 1999; Whitehead et al., 2001b).

No significant decreases in OdDHL levels were seen with either the crude extract or the purified compounds (Table 2), the former being consistent with the data reported previously (Adonizio et al., 2008b). BHL levels however, were affected by the crude extract at a concentration of 1 mg/ml (20% reduction) and purified vescalagin (21% reduction). This is consistent with our previous work on C. erectus which revealed a 25% decrease in BHL levels, suggesting that vescalagin is responsible for this reduction.

TABLE 2
Effect of ellagitannins on P. aeruginosa QS genes and AHL production.

| | | | Gene Expression (measured via β-galactosidase activity | | | |
|---|---|---|---|---|---|---|
| Culture | AHL production (μM) | | of the lacZ fusion products and expressed as Milller Units) | | | |
| Conditions | C12-AHL | C4-AHL | las1 | lasR | rhll | rhlR |
| Media only | 1.328 ± 0.14 | 0.621 ± 0.03 | 3873 ± 260 | 4823 ± 385 | 4933 ± 333 | 7639 ± 216 |
| Crude 1 mg/ml | 1.082 ± 0.07 | 0.496 ± 0.05 | 3065 ± 247 | 1325 ± 275 | 3383 ± 111 | 4535 ± 231 |
| Crude 40 μg/ml | 1.431 ± 0.06 | 0.578 ± 0.08 | 3367 ± 276 | 2873 ± 188 | 3514 ± 306 | 4129 ± 287 |
| Vescalagin | 1.227 ± 0.16 | 0.489 ± 0.01 | 2049 ± 93 | 1703 ± 297 | 3514 ± 306 | 4129 ± 287 |
| Castalagin | 1.389 ± 0.20 | 0.703 ± 0.01 | 2465 ± 116 | 2160 ± 509 | 4542 ± 199 | 4904 ± 348 |

Ellagitannins Affect QS Gene Expression.

P. aeruginosa elaborates two main sets of QS systems: lasI-lasR and rhlI-rhlR (Schuster & Greenberg, 2006). LasI and RhlI are synthetases that manufacture the autoinducer signaling molecules mentioned in the previous section. The receptors, lasR and rhlR, are activated by these signals and, in turn, coordinate the regulation of pathogenicity.

The effect of castalagin and vescalagin on the transcriptional activity of the QS-gene promoters was tested using PAO1-derived strains harboring promoter-lacZ fusions (as described in (Kong et al., 2005) and (Adonizio et al., 2008b). Assays for β-galactosidase (lacZ) activity in P. aeruginosa were performed with o-nitrophenyl-β-D-galactopyranoside, as described previously (Mathee et al., 1997).

Significant effects were seen on the activity of all tested QS genes with C. erectus at 1 mg/ml (Table 2) which agrees with previous results on this species (Adonizio et al., 2008b). Crude extract at 40 μg/ml reduced the expression of all QS genes to a lesser extent, leaving lasI marginally insignificant. Castalagin reduced all QS gene levels save for rhlI which may correspond to the lack of effect on its signaling molecule OdDHL (Table 1). Vescalagin on the other hand reduced all QS gene levels including rhlI to a greater extent than castalagin. The significant reduction of rhlI by vescalagin may correspond to its effect on BHL.

Interestingly, reduction of lasI is more pronounced with the addition of pure compounds than with the crude extract (Table 2). This may suggest another compound in the crude extract causing up-regulation of lasI or simply blocking its repression by the ellagitannins thus balancing out the effect.

Ellagitannins do not Affect Bacterial Growth.

To confirm that the reduction in virulence was due to QS inhibition and not static or cidal effects, cell proliferation was monitored using growth curve studies and the Bradford assay (Bradford, 1976). No significant differences in growth of PAO1 were seen with either concentration of crude extract or the purified compounds. In comparison, previous work on ellagic acid shows no effect on growth of the related species P. putida at concentrations up to 30 μg/ml (Huber et al., 2004).

### CONCLUSION

Previous research on C. erectus (Adonizio et al., 2008a; Adonizio et al., 2008b, the disclosures of which are incorporated herein by reference in their entireties) indicates a marked reduction of QS and inhibition of P. aeruginosa virulence. The activity of the isolated compounds vescalagin and castalagin demonstrated herein account for the majority of the activity in this plant, suggesting a new mode of action for ellagitannins. Purified ellagitannins affected AHL levels, and QS-gene expression similarly to the parent extract. Protease and elastase levels were also markedly reduced, however, pyoverdin was not affected. This may suggest that additional compounds within C. erectus are responsible for the entire anti-QS effect or that the regulation of pyoverdin extends beyond the QS system.

The latter hypothesis is supported by previous research on the nature of pyoverdin (Beare et al., 2003; Lamont et al., 2002, the disclosures of which are incorporated herein by reference in their entireties) and the mixed results of halogenated furanones on its production (Hentzer et al., 2003; Sakar et al., 2005, the disclosures of which are incorporated herein by reference in their entireties).

In several cases (Table 1 and 2), differences in the degree of anti-QS activity between the crude extract and the purified compounds can be explained by a dosage effect. The extraction of vescalagin and castalagin from the crude extract was found to be roughly 6% in both cases with the methods exemplified herein. Thus, the working concentration of ellagitannins is as follows: 60 μg/ml in the 1 mg/ml crude extract and 2.4 μg/ml in the 40 μg/ml crude extract. The tested concentration of the purified compounds falls between these two as does the activity in most cases.

Castalagin and vescalagin have not been previously isolated from Conocarpus erectus, and although they may be part of a larger C-glycosidic ellagitannin oligomer, it is likely these compounds exist in their native state as well. Although tannins are widespread throughout the plant kingdom, NHTP-bearing ellagitannins such as castalagin and vescalagin are mostly limited to the Combretaceae, Fagaceae, Melastomataceae, and Myrtaceae (Okuda et al., 2000, the disclosure of which is incorporated herein by reference in its entirety). Interestingly, the plant species found in our previous work to have the highest levels of anti-QS activity are within these four families Conocarpus erectus L. (Combretaceae), Callistemon viminalis (Sol. ex Gaertn.) G. Don (Myrtaceae), Bucida burceras L. (Combretaceae), Tetrazygia bicolor (Mill.) Cogn. (Melastomataceae), and Quercus virginiana Mill. (Fagaceae). (Adonizio et al., 2008b, the disclosure of which is incorporated herein by reference in its entirety). The data presented herein suggest that similar ellagitannins may be responsible for QS inhibition in these plants.

For the sake of completeness of disclosure, all patent documents and literature articles cited herein are expressly incorporated in this specification by reference in their entireties.

The foregoing description and Example has been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

### REFERENCES

Adonizio, A. Jr., S. M. L., Ausubel, F. M. & Mathee, K. (2008a). Medicinal plant extracts attenuate Pseudomonas aeruginosa killing of Caenorhabditis elegans through bacterial virulence inhibition. Journal of Medical Microbiology (In Press)
Adonizio. A., Kong. K.-F. & Mathee, K. (2008b). Inhibition of quorum sensing-controlled virulence factor production in Pseudomonas aeruginosa by south Florida plant extracts. Antimicrobial Agents and Chemotherapy 52, 198-203.
Adonizio, A. L. Downum, K., Bennett, B. C. & Mathee, K. (2006). Anti-quorum sensing activity of medicinal plants in southern Florida. Journal of Ethnopharmacology 103, 427-435.
Albus, A., Pesci, E. Runyen-Janecky, L. West, S. & lglewski, B. (1997). Vfr controls quorum sensing in Pseudomonas aeruginosa. Journal of Bacteriology 179, 3928-3935.
Bauer. A. W., Kirby, W. M., Sherris, J. C. & Turck, M. (1966). Antibiotic susceptibility testing by a standardized single disk method. American, Journal of Clinical Pathology 45, 493-496.
Bauer. W. D. & Mathesius. U. (2004). Plant responses to bacterial quorum sensing signals. Current Opinion in Plant Biology 7, 429-433.
Bauer. W. D., Tepletski, M. (2001). Can plants manipulate bacterial quorum sensing? Australian Journal of Plant Physiology 28, 913-921.
Bodey, G. P., Bolivar, R. Fainstein, V. & Jadeja, L. (1983). Infections caused by Pseudomonas aeruginosa. Reviews of Infectious Diseases 5, 279-313.
Bradford, M. M. (1976). A rapid and sensitive method for the quantitation of microgram quantities of protein utilizing the principle of protein-dye binding Analytical Biochemistry 72, 248-254.
Calfee, M. W. Coleman. J. P. & Pesci, E. C. (2001). Interference with Pseudomonas quinolone signal synthesis inhibits virulence factor expression by Pseudomonas aeruginosa. Proceedings of the National Academy of Sciences of the United States of America 98, 11633-11637.
Cowan. M. M. (1999). Plant products as antimicrobial agents. Clinical Tlicrobio Reviews 12, 564-582.
Cox. C. D. & Adams, P. (1985). Siderophore activity of pyoverdin for Pseudomonas aeruginosa. Infection and Immunity 48, 130-138.
Cumberbatch, A. (2002). Characterization of the anti-quorum sensing activity exhibited by marine macroalgae of South Florida. In Department of Biological Sciences. Miami: Florida International University.
Davies, D. G., Parsek, M. R., Pearson. J. P. Iglewski, B. H., Costerton. J. W. & Greenberg, E. P. (1998). The involvement of cell-to-cell signals in the development of a bacterial biofilm. Science 280, 295-298.
Dong, Y. H. Wang, L. H., Xu, J. L., Zhang, H. B., Zhang, X. F. & Zhang, L. H. (2001). Quenching quorum-sensing-dependent bacterial infection by an N-acyl homoserine lactonase. Nature 411, 813-817.
Dong, Y. H. & Zhang, L. H. (2005). Quorum sensing and quorum-quenching enzymes. Journal of Microbiology 43 Spec No, 101-109.
Donlan. R. M. & Costerton, J. W. (2002). Biofilms: survival mechanisms of clinically relevant microorganisms. Clinical Microbiology Reviews 15.167-193.
Duke. J. A. (1985). CRC Handbook of Medicinal Herbs. Boca Raton, Fla.: CRC Press.
Fuqua, C. & Greenberg, E. P. (1998). Self perception in bacteria: quorum sensing with acylated homoserine lactones. Current Opinion in Microbiology 1, 183-189.
Fuqua, C. & Greenberg, E. P. (2002). Listening in on bacteria: acyl-homoserine lactone signalling. Nature Reviews Molecular and Cellular Biology 3.685-695.
Fuqua, W. C., Winans. S. C. & Greenberg, E. P. (1994). Quorum sensing in bacteria: the LuxR-Luxi family of cell density-responsive transcriptional regulators. Journal of Bacteriolog y 176.269-275.
Gallagher, L. A. & Manoil, C. (2001). Pseudomonas aeruginosa PAOI kills Caenorhanditis elegans by cyanide poisoning. Journal of Bacteriology 183, 6207-6214.
Galleni. M. Lindberg, F., Normark, S. Cole, S., Honore, N., Joris, B. & Frere, J. M. (1988). Sequence and comparative analysis of three Enterobacter cloacae ampC betalactamase genes and their products. Biochemical Journal 250, 753-760.
Gambello, M. J. Iglewski, B. H (1991). Cloning and characterization of the Pseudononas aeruginosa lasR gene, a transcriptional activator of elastase expression. Journal of Bacteriology 179, 3000-3009.
Garg, S. C. & Kasera, H. L. (1982). Antihelminthic activity of the essential oil o Callisternon vim/nails. Fitotercrpia 53, 179-181.
Givskov. M., de Nys, R., Manefield, M., Gran), L., Maximilien, R. Eberl, L., Molin, S., Steinberg. P. D. & Kjelleberg. S. (1996). Eukaryotic interference with homoserine lactone-mediated prokaryotic signalling. Journal of Bacteriology 178, 6618-6622.
Hastings, J. W. & Greenberg, E. P. (1999). Quorum sensing: the explanation of a curious phenomenon reveals a common characteristic of bacteria. Journal of Bacteriology 181. 2667-2668.
Heijden, R., Jacobs, D. 1., Snoeijer, W., Hallard, D. & Verpoorte. R. (2004). The lrus alkaloids: pharmacognosy and biotechnology. Current Medicinal Chemistry 1 I. 607-628.
Hentzer, M. Reidel, K. Rasmussen, T. B. & other authors (2002). Inhibition of quorum sensing in Pseudonronas aeruginosa biofilm bacteria by a halogenated furanone compound. Microbiology 148, 87-102.
Hentzer. M. & Givskov, M. (2003a). Pharmacological inhibition of quorum sensing for the treatment of chronic bacterial infections. Journal of Clinical Investigation 112, 1300-1307.
Hentzer, M. & Givskov, M. (2003b). Pharmacological inhibition of quorum sensing for the treatment of chronic bacterial infections. Jour not of Clinical Investigation 112. 1300-1307.
Hentzer, M. Wu, H., Andersen, J. B. & other authors (2003). Attenuation of Pseudonronas aeruginosa virulence by quorum sensing inhibitors. The Et1'IB0 Journal 22. 3803-3815.
Hersch-Martinez. P., Leanos-Miranda, B. E. & Solorzano-Santos, F. (2005). Antibacterial effects of commercial essential oils over locally prevalent pathogenic strains in Mexico. Fztoterapra 76, 453-457.
Holden et al., (1999). Quorum-sensing cross talk: isolation and chemical characterization of cyclic dipeptides from Pseudomonas aeruginosa and other Gram-negative bacteria. Molecular Microbiology 33, 1254-1266.
Holloway, B. W. & Morgan, A. F. (1986). Genome organization in Pseudomonas. Annual Review of Microbiology 40, 79-105.
Hoyle, B. & Costerton, J. W. (1991). Bacterial resistance to antibiotics: the role of biofilms. Progress in Drug Research 37, 91-105.
Huber, B. Ebert, L., Feucht. W. & Polster, J. (2004). Influence of polyphenols on bacterial biofilm formation and quorum-sensing. Zeitschrift fur Naturforschung Section C, Biosciences 58, 879-884.
Kessler, E. (1995). Beta-lytic endopeptidases. Methods in Enzymology 248, 740-756.
Khanbabaee K. & van Ree, T. (2001). Tannins: classification and definition. Natural Product Reports 18, 641-649.
Kharazmi, A. (1989). Interactions of Pseudomonas aeruginosa proteases with the cells of the immune system. Antibiotics and Chemotherapy 42, 42-49.
Kemery. V., Koprnova, J. Gogova, M., Grey, E. & Korcova, J. (2006). Pseudomonas aeruc ino.sa bacteraemia in cancer patients., 16urnal of Infection 52.461-463.
Manefield, M., de Nys. R. Kumar. N. Read, R. Givskov, M., Steinberg. P. & Kjelleberg, S. (1999). Evidence that halogenated furanones from Delisea pulchra inhibit acylated homoserine lactone (AHL)-mediated gene expression by displacing the AHL signal from its receptor protein. Microbiology 145, 283-291.
Manefield, M. 1 asmussen. B. I Ienzter, M. Andersen. J. B. Steinberg. P., Kjelleberg, S. & (ii\skov, lbl. (2002). Halogenated furanones inhibit quorum sensing through accelerated I uxR turnover. Microbiology 148, 1119-1 127.
Melendez, E. N. (1982). Plantas medicinales de Puerto Rico: folklore y fundementos cientificos. Rio Piedras, P. R.: Editorial de la Unversidad de Puerto Rico.
Morihara. K. & Homma. J. Y. (1985). Pseudomonas Proteases. In Bacterial Enzymes and Virulence, pp. 41-79. Edited by I. Holder. Boca Raton, Fla.: CRC Press, Inc.
Ohman. D. E. Cryz, S. J. & Iglewski, B. Fl. (1980). Isolation and characterization of a Pseudomonas aeruginosa PAO mutant that produces altered elastase. Journal of Bacteriology 142, 836-842.
Okuda. T., Yoshida, T. & Hatano, T. (1993). Classification of oligomeric hydrolysable tannins and specificity of their occurrence in plants. Phvtochemistry 32, 507-521.
Okuda. T. Yoshida. T. & Hatano, T. (2000). Correlation of oxidative transformations of hydrolyzable tannins and plant evolution. Phytochemi.stry 55, 513-529.
Oszmianski, J., Ramos. T. & Bourzeix, M. (1988). Fractionation of phenolic compounds in red wine. American Journal of Enology and awry ire 39, 259-262.
Pesci. E. Pearson, J., Seed, P. & 1˜glewski. B. (1997). Regulation of/as and rhl quorum sensing in Pseudomonas aeruginosa . Jour-no/of Bacteriology 179, 3127-3132.
Pesci et al., (1999). Quinolone signaling in the cell-to-cell communication system of Pseudomonas aeruginosa. Proceedings of the National Academy of Sciences of the United States of America 96, 11229-11234.
Pessi, G. & Haas. D. (2000). Transcriptional control of the hydrogen cyanide biosynthetic genes hcnABC by the anaerobic regulator ANR and the quorum-sensing regulators LasR and RhIR in Pseudomonas aeruginosa. Journal of Bacteriology 182, 6940-6949.
Quideau, S. & Feldman, K. S. (1996). Ellagitannin chemistry-. Chemical Reviews 96, 475-504.
Smith, R. S. & Iglewski. B. H. (2003a). Pseudomonas aeruginosa quorum sensing as a potential antimicrobial target. Journal of Clinical Investigation 112, 1460-1465.
Smith. R. S. & Iglewski, B. H. (2003b). Pseudomonas aeruginosa quorum-sensing systems and virulence. Current Opinion in Microbiology 6, 56-60.
Smith. R. S. & Iglewski. B. H. (2003c). Pseudomonas aeruginosa quorum sensing as a potential antimicrobial target. Journal of Clinical Investigation 1 12. 1460-1465.
Sun, B. Leandro. M. C., de Freitas, V. & Spranger, M. 1. (2006). Fractionation of red wine polyphenols by solid-phase extraction and liquid chromatography. Journal of ChromatographyA 1128.27-38.
Surette, M. G. & Bassler, B. L. (1998). Quorum sensing in Escherichia coli and Salmonella typhimurium. Proceedings of the National Academy of Sciences of the United States of America 95. 7046-7050.
Tan. M. W. Mahajan-Miklos S & Ausubel. F. M. (1999). Killing of Caenorhabditis elegans by Pseudomonas aeruginasa used to model mammalian bacterial pathogenesis. Proceedings of the National Academy of Sciences of the United States of America 96, 715-720.
Tang and Hancock. R. A. (1995). Studies of vegetable tannins. Complete structural determination of two chestnut tannins-vescalagin and eastalagin using nuclear magnetic resonance spectroscopic methods. Journal of the Society of Leather Technologists and Chemists, 79:181-187, 1995.
Zhang, L. H. & Dong, Y. H. (2004). Quorum sensing and signal interference: diverse implications. Molecular Microbiology 53, 1563-1571.
Zhentian. L., Jervis. J. & Helm, R. F. (1999). C-Glycosidic ellagitannins from white oak heartwood and callus tissues. Phytochemistry 51, 751-756.
Zhu M., David Phillipson, J., Greengrass, P. M. Bowery. N. F. & Cal. Y. (1997). Plant polyphenols: Biologically active compounds or non-selective binders to protein? Photochemistry 44, 441-447.

1. A method of inhibiting quorum sensing in bacteria comprising contacting the bacteria with an isolated ellagitannin in an amount effective to inhibit quorum sensing in said bacteria, wherein the ellagitannin is selected from the group consisting of vescalagin, castalagin, Rhoipteleanin H, Rhoipteleanin J, stachyurin, casuarinin, 5-desgalloyl-stachyurin, casuariin, roburin A, roburin D, cercidinin A, cercidinin B and grandinin.

2. The method of claim 1, wherein the bacteria is contacted with the isolated ellagitannin in vivo.

3. The method of claim 2, wherein the contacting comprises administering the isolated ellagitannin to a mammalian subject.

4. The method of claim 3, wherein the mammalian subject is human.

5. The method of claim 3, wherein the human is immunocompromised.

6. The method of claim 3, wherein the mammalian subject is afflicted with a bacterial infection associated with quorum sensing and the isolated ellagitannin is administered in an amount effective to treat the bacterial infection.

7. The method of claim 3, wherein the mammalian subject is afflicted with a disorder associated biofilm formation and the isolated ellagitannin is administered in an amount effective to treat the disorder.

8. The method of claim 6, wherein the mammalian subject is resistant to therapy with a standard of care anti-bacterial therapeutic.

9. The method of claim 1, wherein the bacteria is contacted with the isolated ellagitannin ex vivo.

10. The method of claim 9, wherein the contacting comprises administering the ellagitannin to an inanimate surface.

11. method of claim 10, wherein the inanimate surface is a medical device and the isolated ellagitannin is applied to the device in an amount effective to inhibit biofilm formation associated with bacterial quorum sensing on a medical device.

12. The method of claim 1, further comprising contacting the bacteria with an anti-bacterial agent.

13. (canceled)

14. A method of treating a disorder associated with biofilm formation in a mammalian subject comprising administering to the subject an isolated ellagitannin in an amount effective to treat the disorder, wherein the ellagitannin is selected from the group consisting of vescalagin, castalagin, Rhoipteleanin H, Rhoipteleanin J, stachyurin, casuarinin, 5-desgalloyl-stachyurin, casuariin, roburin A, roburin D, cercidinin A, cercidinin B and grandinin.

15. The method claim 13, further comprising administering a standard of care anti-bacterial therapeutic to the subject.

16. A method of treating a disorder associated with quorum sensing in a mammalian subject resistant to treatment with a standard of care anti-bacterial therapeutic comprising administering to the subject an isolated ellagitannin in an amount effective to treat the disorder, wherein the ellagitannin is selected from the group consisting of vescalagin, castalagin, Rhoipteleanin H, Rhoipteleanin J, stachyurin, casuarinin, 5-desgalloyl-stachyurin, casuariin, roburin A, roburin D, cercidinin A, cercidinin B and grandinin.

17. (canceled)

18. The method of claim 15, wherein the standard of care anti-bacterial therapeutic is selected from the group consisting of colloidal silver, penicillin, penicillin G, erythromycin, polymyxin B, viomycin, chloromycetin, streptomycins, cefazolin, ampicillin, methicillin, oxacillin, nafcillin, cloxacillin, dicloxacillin azactam, tobramycin, cephalosporins, carbapenemsbacitracin, tetracycline, doxycycline, gentamycin, quinolines, neomycin, clindamycin, kanamycin, metronidazole, treptogramins, Streptomycin, Ceftriaxone, Cefotaxime, Rifampin, vancomycin, teicoplanin, Ortivancin, erythromycin, clarithromycin, azithromycin, lincomycin, clindamycun, Telithromycin, ABT-773, Tetracyclines, Terbutyl-minocycline (GAR-936)), Aminoglycosides, Chloramphenicol, Imipenem-cilastatin, dalbavanci), ofloxacin, sparfioxacin, gemifloxacin, cinafloxacun (DU-6859a), Trimethoprim-sulfamethoxazole (TMP-SMX), Ciprofloxacin, topical mupirocin, AZD-2563, Linezolid (Zyvox™)), Daptomycin, Ramoplanin), ATD-6424 (Theravance), isoniazid (INN), pyrazinamide (PZA), Ethambutol (EMB), Capreomycin, cycloserine, ethionamide (ETH), kanamycun, and p-aminosalicylic acid (PAS).

19. (canceled)

20. The method of claim 1, wherein the isolated ellagitannin is vescalagin or castalagin.

21. (canceled)

22. The method of claim 1, wherein the bacteria is of a genus selected from the group consisting of Aeromonas, Agrobacterium, Burkholderia, Chromobacterium, Enterobacter, Erwinia, Escherichia, Nitrosomas, Obesumbacterium, Pantoea, Pseudomonas, Ralstonia, Rhisobium, Rhodobacter, Serratia, Staphylococcus, Vibrio, Xenorhabdus, and Yersinia.

23. The method of claim 1, wherein the bacteria is of a species selected from the group consisting of Aeromonas hydrophila, Aeromonas salmonicida, Agrobacterium tumefaciens, Burkholderia cepacia, Chromobacterium violaceum, Enterobacter agglomeran, Erwinia carotovora, Erwinia chrysanthemi, Escherichia coli, Nitrosomas europaea, Obesumbacterium proteus, Pantoea stewartii, Pseudomonas aureofaciens, Pseudomonas aeruginosa, Pseudomonas syringae, Ralstonia solanacearum, Rhisobium etli, Rhisobium leguminosarum, Rhodobacter sphaeroides, Serratia liguefaciens, Serratia marcescens, Staphyllococcus aureus, Staphyllococcus epidermidis, Vibrio anguillarum, Vibrio fischeri, Vibrio cholerae, Xenorhabdus nematophilus, Yersinia enterocolitica, Yersinia pestis, Yersinia pseudotuberculosis, Yersinia medievalis, and Yersinia ruckeri.

24. The method of claim 1, wherein the isolated ellagitannin is present in a composition comprising a pharmaceutically-acceptable carrier, excipient or diluent.

25-26. (canceled)

27. A method of inhibiting biofilm formation on a surface comprising contacting the surface with an isolated ellagitannin in amount effective to inhibit biofilm formation on said surface, wherein the ellagitannin is selected from the group consisting of vescalagin, castalagin, Rhoipteleanin H, Rhoipteleanin J, stachyurin, casuarinin, 5-desgalloyl-stachyurin, casuariin, roburin A, roburin D, cercidinin A, cercidinin B and grandinin.

28. The method of claim 10, wherein the inanimate surface is selected from the group consisting of a metal surface, a glass surface, a plastic surface, a wood surface and a stone surface.
